(19) United States
(12) Patent Application Publication
(10) Pub. No.: US 2017/0137834 A1
(43) Pub. Date: May 18, 2017

(54) ZEA MAYS REGULATORY ELEMENTS AND USES THEREOF

(71) Applicant: Dow AgroSciences LLC, Indianapolis, IN (US)

(72) Inventors: Manju Gupta, Carmel, IN (US); Sandeep Kumar, Carmel, IN (US); Navin Elango, Indianapolis, IN (US); Jeffrey Beringer, Carmel, IN (US); Shavell Gorman, Indianapolis, IN (US); Andrew F. Worden, Indianapolis, IN (US); Sara Bennett, Indianapolis, IN (US); Daren Hemingway, Westfield, IN (US); Wei Chen, Carmel, IN (US); Huixia Wu, Zionsville, IN (US); Ning Zhou, Zionsville, IN (US); Michelle Sprint Smith, Carmel, IN (US)

(73) Assignee: Dow AgroSciences LLC, Indianapolis, IN (US)

(21) Appl. No.: 15/331,126

(22) Filed: Oct. 21, 2016

Related U.S. Application Data
(60) Provisional application No. 62/244,369, filed on Oct. 21, 2015.

(51) Int. Cl. C12N 15/82; C07K 14/325

(52) U.S. Cl. CPC C12N 15/8227; C12N 15/8241; C12N 15/8286; C12N 15/8274; C12N 15/8273; C12N 15/8242; C07K 14/325

(57) ABSTRACT

Provided are constructs and methods for expressing a transgene in plant cells and/or plant tissues using Zea mays GRMZM2G015295 gene regulatory elements.

### CROSS REFERENCE TO RELATED APPLICATION

The present application claims priority to the benefit of U.S. Provisional Patent Application Ser. No. 62/244,369 filed Oct. 21, 2015 the disclosure of which is hereby incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: one 30.7 KB ACII (Text) file named “76229-US-PSP-20151020-Sequence-Listing-ST25.txt” created on Oct. 20, 2015.

### FIELD OF THE INVENTION

This invention is generally related to the field of plant molecular biology, and more specifically, to the field of expression of transgenes in plants.

### BACKGROUND

Recombinant DNA technology and genetic engineering have made it routinely possible to introduce desired DNA sequences into plant cells to allow for the expression of proteins of interest. For commercially viable transformation events, however, obtaining desired levels of stable and predictable expression in important crops remains challenging.

Transgenic plant species comprising multiple transgenes stacked at a single genomic locus are produced via plant transformation technologies. Plant transformation technologies result in the introduction of a transgene into a plant cell, recovery of a fertile transgenic plant that contains the stably integrated copy of the transgene in the plant genome, and subsequent transgene expression via transcription and translation of the plant genome results in transgenic plants that possess desirable traits and phenotypes. However, mechanisms that allow the production of transgenic plant species to highly express multiple transgenes engineered as a trait stack are desirable.

One method of expressing heterologous genes at desired levels in crops involves manipulation of the regulatory mechanisms governing expression in plants. The regulation may be transcriptional or post-transcriptional and can include, for example, mechanisms to enhance, limit, or prevent transcription of the DNA, as well as mechanisms that limit or increase the life span of an mRNA after it is produced. The DNA sequences involved in these regulatory processes can be located upstream, downstream or even internally to the structural DNA sequences encoding the protein product of a gene.

To regulate transcription in a transgenic plant, various types of promoters may be employed. Promoters can be used to control the expression of foreign genes in transgenic plants in a manner similar to the expression pattern of the gene from which the promoter was originally derived. In general, promoters are classified in two categories: “Constitutive” promoters express in most tissues most of the time, while “regulated” promoters are typically expressed in only certain tissue types (tissue specific promoters) or at certain times during development (temporal promoters). Expression from a constitutive promoter is typically more or less at a steady state level throughout development. Genes encoding proteins with house-keeping functions are often driven by constitutive promoters. Examples of constitutively expressed genes in maize include actin and ubiquitin.

Further improvements in transcription can be obtained in transgenic plants by placing “enhancer” sequences upstream (5′) of the promoter. Enhancer elements are cis-acting and increase the level of transcription of an adjacent gene from its promoter in a fashion that is relatively independent of the upstream position and orientation of the enhancer. Such sequences have been isolated from a variety of sources, including viruses, bacteria and plant genes. One example of a well characterized enhancer sequence is the octopine synthase (ocs) enhancer from the Agrobacterium tumefaciens, as described in U.S. Pat. Nos. 5,837,849, 5,710,267 and 5,573,932. This short (40 bp) sequence has been shown to increase gene expression in both dicots and monocots, including maize, by significant levels. Tandem repeats of this enhancer have been shown to increase expression of the GUS gene eight-fold in maize. It remains unclear how these enhancer sequences function. Presumably enhancers bind activator proteins and thereby facilitate the binding of RNA polymerase II to the TATA box. WO95/14098 describes testing of various multiple combinations of the ocs enhancer and the mas (mannopine synthase) enhancer which resulted in several hundred fold increase in gene expression of the GUS gene in transgenic tobacco callus.

The use of a specific promoter, with or without one or more enhancers, however, does not necessarily guarantee desired levels of gene expression in plants. In addition to desired transcription levels, other factors such as improper splicing, polyadenylation and nuclear export can affect accumulation of both mRNA and the protein of interest. Therefore, methods of increasing RNA stability and translational efficiency through mechanisms of post-transcriptional regulation are needed in the art.

With regard to post-transcriptional regulation, it is has been demonstrated that certain 5′ and 3′ untranslated regions (UTRs) of eukaryotic mRNAs play a major role in translational efficiency and RNA stability, respectively. For example, the 5′ and 3′ UTRs of tobacco mosaic virus (TMV) and alfalfa mosiac virus (AMV) coat protein mRNAs are known to enhance gene expression in tobacco plants. The 5′ and 3′ UTRs of the maize alcohol dehydrogenase-1 (adh1) gene are known to be involved in efficient translation in hypoxic protoplasts. Additionally, the use of diversified regulatory elements, especially in multigene molecular stack constructs have been pivotal to consistent and sustained expression of transgenes in order to minimize homology-based gene silencing (Peremarti et al., 2010; Mourrain et al., 2007; Bhullar et al., 2003).

Experiments with various 5′ UTR leader sequences demonstrate that various structural features of a 5′ UTR can be correlated with levels translational efficiency. Certain 5′ UTRs have been found to contain AUG codons which may interact with 40 S ribosomal subunits when it scans for the AUG codon at the initiation site, thus decreasing the rate of translation. (Kozak, Mol. Cell. Biol. 7:3438 (1987); Kozak, J. Cell Biol. 108, 209 (1989)). Further, the 5′ UTR nucleotide sequences flanking the AUG initiation site on the mRNA may have an impact on translational efficiency. If the context of the flanking 5′ UTR is not favorable, part of the 40 S ribosomal subunits might fail to recognize the translation start site such that the rate of polypeptide synthesis will be slowed. (Kozak, J. Biol. Chem. 266, 19867-19870 (1991); Pain, Eur. J. Biochem. 236, 747-771 (1996)). Secondary structures of 5′ UTRs (e.g., hairpin formation) may also hinder the movement of 40 S ribosomal subunits during their scanning process and therefore negatively impact the efficiency of translation. (Sonenberg et al., Nature 334:320 (1988); Kozak, Cell 44:283-292, (1986)). The relative GC content of a 5′ UTR sequence has been shown to be an indicator of the stability of the potential secondary structure, with higher levels of GC indicating instability. (Kozak, J. Biol. Chem. 266, 19867-19870 (1991). Longer 5′ UTRs may exhibit higher numbers of inhibitory secondary structures. Thus, the translational efficiency of any given 5′ UTR is highly dependent upon its particular structure, and optimization of the leader sequence has been shown to increase gene expression as a direct result of improved translation initiation efficiency. Furthermore, significant increases in gene expression have been produced by addition of leader sequences from plant viruses or heat shock genes. (Raju et al., Plant Science 94: 139-149 (1993)).

In addition to 5′ UTR sequences, 3′ UTR (trailer) sequences of mRNAs are also involved in gene expression. 3′ UTRs (also known as polyadenylation elements or adenylation control elements) are known to control the nuclear export, polyadenylation status, subcellular targeting and rates of translation and degradation of mRNA from RNases. In particular, 3′ UTRs may contain one or more inverted repeats that can fold into stem-loop structures which act as a barrier to exoribonucleases, as well as interact with proteins known to promoter RNA stability (e.g., RNA binding proteins). (Barkan et al., A Look Beyond Transcription: Mechanisms Determining mRNA Stability and Translation in Plants, American Society of Plant Physiologists, Rockville, Md., pp. 162-213 (1998)). Certain elements found within 3′ UTRs may be RNA destabilizing, however. One such example occurring in plants is the DST element, which can be found in small auxin up RNAs (SAURs). (Gil et al., EMBO J. 15, 1678-1686 (1996)). A further destabilizing feature of some 3′ UTRs is the presence of AUUUA pentamers. (Ohme-Takagi et al., Pro. Nat. Acad. Sci. USA 90 11811-11815 (1993)).

3′ UTRs have been demonstrated to play a significant role in gene expression of several maize genes. Specifically, a 200 base pair 3′ sequence has been shown to be responsible for suppression of light induction of the maize small m3 subunit of the ribulose-1,5-biphosphate carboxylase gene (rbc/m3) in mesophyll cells. (Viret et al., Proc Natl Acad Sci USA. 91 (18):8577-81 (1994)). In plants, especially maize, this sequence is not very well conserved. One 3′ UTR frequently used in genetic engineering of plants is derived from a nopaline synthase gene (3′ nos) (Wyatt et al., Plant Mol Biol 22(5):731-49 (1993)).

In certain plant viruses, such as alfalfa mosaic virus (AMV) and tobacco mosaic virus (TMV), their highly structured 3′ UTRs are essential for replication and can be folded into either a linear array of stem-loop structures which contain several high-affinity coat protein binding sites, or a tRNA-like site recognized by RNA-dependent RNA polymerases. (Olsthoorn et al., EMBO J 1; 18(17):4856-64 (1999); Zeyenko et al., 1994)).

However, there remains a need to identify additional 5′ and 3′ UTRs for their use in regulating expression of recombinant nucleic acids in transgenic plants because there are no optimal UTR sequences available for every application.

### SUMMARY OF THE INVENTION

Disclosed herein are sequences, constructs, and methods for expressing a transgene in plant cells and/or plant tissues. In an embodiment the disclosure relates to a gene expression cassette comprising a promoter operably linked to a transgene, wherein the promoter comprises a polynucleotide that hybridizes under stringent conditions to a polynucleotide probe comprising a sequence identity of at least 90% to a complement of SEQ ID NO:1. In further embodiments, the promoter comprises a polynucleotide that has at least 90% sequence identity to SEQ ID NO:1. In an embodiment, the promoter comprises a polynucleotide that has at least 90% sequence identity to SEQ ID NO:2. In other embodiments, the operably linked transgene encodes a polypeptide or a small RNA. In a subsequent embodiment, the transgene is selected from the group consisting of insecticidal resistance transgene, herbicide tolerance transgene, nitrogen use efficiency transgene, water use efficiency transgene, nutritional quality transgene, DNA binding transgene, and selectable marker transgene. In yet another embodiment, the gene expression cassette further comprises a 3′-untranslated region. In an embodiment the 3′-untranslated region comprises a polynucleotide that has a sequence identity of at least 90% to SEQ ID NO:3 or SEQ ID NO:4. In an embodiment, a recombinant vector comprises the gene expression cassette. In a further aspect of the embodiment, the recombinant vector is selected from the group consisting of a plasmid, a cosmid, a bacterial artificial chromosome, a virus, and a bacteriophage. In an embodiment, a transgenic cell comprises the gene expression cassette. In a subsequent aspect of the embodiment, the cell is a transgenic plant cell. In an embodiment, a transgenic plant comprises the transgenic plant cell. In a further aspect of the embodiment, the transgenic plant is a monocotyledonous plant or dicotyledonous plant. In other aspects of the embodiment, the monocotyledonous plant is selected from the group consisting of a maize plant, a rice plant, and a wheat plant. In an embodiment, a transgenic seed is obtained from the transgenic plant. In a subsequent embodiment, the promoter is a tissue-preferred promoter. In an additional embodiment, the tissue-preferred promoter is a root tissue-preferred promoter. In yet another embodiment, the promoter comprises a polynucleotide sequence of nucleotides 1-2,089 of SEQ ID NO:1.

In an embodiment the disclosure relates to a transgenic cell comprising a synthetic polynucleotide that hybridizes under stringent conditions to a polynucleotide probe comprising a sequence identity of at least 90% to a complement of SEQ ID NO:1. In an additional embodiment, the synthetic polynucleotide has at least 90% sequence identity to SEQ ID NO:1. In an embodiment, the synthetic polynucleotide comprises a polynucleotide with at least 90% sequence identity to SEQ ID NO:2. In a further embodiment, the transgenic cell is a transgenic plant cell. In a subsequent embodiment, the transgenic plant cell is produced by a plant transformation method. In an additional embodiment, the plant transformation method is selected from the group consisting of an Agrobacterium-mediated transformation method, a biolistics transformation method, a silicon carbide transformation method, a protoplast transformation method, and a liposome transformation method. In an embodiment, a transgenic plant comprises the transgenic plant cell. In a further embodiment, the transgenic plant is a monocotyledonous plant or dicotyledonous plant. In other embodiments, the monocotyledonous plant is selected from the group consisting of a maize plant, a rice plant, and a wheat plant. In an embodiment, a transgenic seed is obtained from the transgenic plant. In an additional embodiment, the promoter is a tissue-preferred promoter. In a subsequent embodiment, the tissue-preferred promoter is a root tissue-preferred promoter. In another embodiment, the synthetic polynucleotide comprises a polynucleotide sequence of nucleotides 1-2,089 of SEQ ID NO:1.

In an embodiment the disclosure relates to a purified polynucleotide promoter, wherein the promoter comprises a polynucleotide that hybridizes under stringent conditions to a polynucleotide probe comprising a sequence identity of at least 90% to a complement of SEQ ID NO:1. In further embodiments, the purified polynucleotide promoter has at least 90% sequence identity to SEQ ID NO:1. In an embodiment, the purified polynucleotide promoter comprises a polynucleotide with at least 90% sequence identity to SEQ ID NO:2. In another embodiment, the purified polynucleotide is operably linked to a transgene. In a subsequent embodiment, the operably linked transgene encodes a polypeptide or is a small RNA. In an embodiment, a gene expression cassette comprises the purified polynucleotide sequence operably linked to the transgene, which is operably linked to a 3′-untranslated region. In an embodiment the 3′-untranslated region comprises a polynucleotide that has a sequence identity of at least 90% to SEQ ID NO:3 or SEQ ID NO:4. In another embodiment, the transgene is selected from the group consisting of insecticidal resistance transgene, herbicide tolerance transgene, nitrogen use efficiency transgene, water use efficiency transgene, nutritional quality transgene, DNA binding transgene, and selectable marker transgene. In an embodiment, a recombinant vector comprises the gene expression cassette. In an additional embodiment, the recombinant vector is selected from the group consisting of a plasmid vector, a cosmid vector, and a BAC vector. In an embodiment, a transgenic cell comprises the gene expression cassette. In a subsequent embodiment the transgenic cell is a transgenic plant cell. In an embodiment, a transgenic plant comprises the transgenic plant cell. In an additional embodiment, the transgenic plant is a monocotyledonous plant. In yet a further embodiment, the monocotyledonous plant is selected from the group consisting of a maize plant, a wheat plant, and a rice plant. In an embodiment, a transgenic seed is obtained from the transgenic plant. In a subsequent embodiment, the purified polynucleotide sequence promotes tissue-preferred expression of a transgene. In an additional embodiment, the purified polynucleotide sequence promotes root tissue-preferred expression of a transgene. In other embodiments, the purified polynucleotide comprises a polynucleotide sequence of nucleotides 1-2,089 of SEQ ID NO:1.

In an embodiment the disclosure relates to a method for expressing a heterologous coding sequence in a transgenic plant, the method comprising:

a) transforming a plant cell with a gene expression cassette comprising a polynucleotide sequence comprising a sequence identity of at least 90% to SEQ ID NO:1 operably linked to the heterologous coding sequence, which is operably linked to a 3′-untranslated region;

b) isolating the transformed plant cell comprising the gene expression cassette;

c) regenerating the transformed plant cell into a transgenic plant; and,

d) obtaining the transgenic plant, wherein the transgenic plant comprises the gene expression cassette comprising the polynucleotide sequence comprising SEQ ID NO:1.

In an additional embodiment, the polynucleotide sequence has at least 90% sequence identity to SEQ ID NO:2. In a further embodiment, the heterologous coding sequence is selected from the group consisting of insecticidal resistance coding sequences, herbicide tolerance coding sequences, nitrogen use efficiency coding sequences, water use efficiency coding sequences, nutritional quality coding sequences, DNA binding coding sequences, and selectable marker coding sequences. In an additional embodiment, transforming of a plant cell utilizes a plant transformation method. In yet another embodiment, the plant transformation method is selected from the group consisting of an Agrobacterium-mediated transformation method, a biolistics transformation method, a silicon carbide transformation method, a protoplast transformation method, and a liposome transformation method. In other embodiments, the transgenic plant is a monocotyledonous transgenic plant or a dicotyledonous transgenic plant. In further embodiments, the monocotyledonous transgenic plant is selected from the group consisting of a maize plant, a wheat plant, and a rice plant. In an embodiment, a transgenic seed is obtained from the transgenic plant. In a further embodiment, the heterologous coding sequence is preferentially expressed in a tissue. In yet another embodiment, the heterologous coding sequence is expressed in a root tissue. In other embodiments, the polynucleotide comprises a sequence of nucleotides 1-2,089 of SEQ ID NO:1.

In an embodiment the disclosure relates to a method for isolating a polynucleotide sequence comprising a sequence identity of at least 90% to SEQ ID NO:1, the method comprising:

e) identifying the polynucleotide sequence comprising a sequence identity of at least 90% to SEQ ID NO:1;

f) producing a plurality of oligonucleotide primer sequences, wherein the oligonucleotide primer sequences bind to the polynucleotide sequence comprising a sequence identity of at least 90% to SEQ ID NO:1;

g) amplifying the polynucleotide sequence comprising a sequence identity of at least 90% to SEQ ID NO:1 from a DNA sample with oligonucleotide primer sequences selected from the plurality of oligonucleotide primer sequences; and,

h) isolating the polynucleotide sequence comprising a sequence identity of at least 90% to SEQ ID NO:1.

In an additional embodiment, the polynucleotide sequence has at least 90% sequence identity to SEQ ID NO:2. In an additional embodiment, the isolated polynucleotide sequence comprising a sequence identity of at least 90% to SEQ ID NO:1 is operably linked to a transgene. In a further embodiment, the operably linked transgene encodes a polypeptide. In an embodiment, a gene expression cassette comprises a polynucleotide sequence with at least 90% sequence identity to SEQ ID NO:1 operably linked to a transgene, wherein the transgene is operably linked to a 3′-untranslated region. In an embodiment the 3′-untranslated region comprises a polynucleotide that has a sequence identity of at least 90% to SEQ ID NO:3 or SEQ ID NO:4. In a further embodiment, the transgene is selected from the group consisting of insecticidal resistance coding sequences, herbicide tolerance coding sequences, nitrogen use efficiency coding sequences, water use efficiency coding sequences, nutritional quality coding sequences, DNA binding coding sequences, and selectable marker coding sequences. In an embodiment, a recombinant vector comprises the gene expression cassette. In a further embodiment, the vector is selected from the group consisting of a plasmid vector, a cosmid vector, and a BAC vector. In an embodiment, a transgenic cell comprises the gene expression cassette. In an additional embodiment, the transgenic cell is a transgenic plant cell. In an embodiment, a transgenic plant comprises the transgenic plant cell. In an additional embodiment, the transgenic plant is a monocotyledonous plant or a dicotyledonous plant. In a further embodiment, the monocotyledonous plant is selected from the group consisting of a maize plant, a wheat plant, and a rice plant. In an embodiment, a transgenic seed is obtained from the transgenic plant. In other embodiments, the isolated polynucleotide comprises a polynucleotie sequence of nucleotides 1-2,089 of SEQ ID NO:1.

In an embodiment the disclosure relates to a method for manufacturing a synthetic polynucleotide sequence comprising a sequence identity of at least 90% to SEQ ID NO:1, the method comprising:
a) identifying the polynucleotide sequence comprising SEQ ID NO:1;
b) isolating the polynucleotide sequence comprising SEQ ID NO:1;
c) defining a plurality of polynucleotide sequences that comprise a sequence identity of at least 90% to SEQ ID NO:1;
d) synthesizing a polynucleotide sequence comprising a sequence identity of at least 90% to SEQ ID NO:1; and,
e) manufacturing a synthetic polynucleotide sequence comprising a sequence identity of at least 90% to SEQ ID NO:1.

In a further embodiment, the synthesizing comprises:
a) identifying the polynucleotide sequence comprising a sequence identity of at least 90% to SEQ ID NO:1;
b) producing a plurality of oligonucleotide primer sequences, wherein the oligonucleotide primer sequences bind to the polynucleotide sequence comprising a sequence identity of at least 90% to SEQ ID NO:1;
c) ligating the plurality of oligonucleotide primer sequences to synthesize the polynucleotide sequence comprising a sequence identity of at least 90% to SEQ ID NO:1.

In an additional embodiment, the synthesized polynucleotide sequence has at least 90% sequence identity to SEQ ID NO:2. In an additional embodiment, the synthesized polynucleotide sequence comprises a sequence identity of at least 90% to SEQ ID NO:1 that is operably linked to a transgene. In yet another embodiment, the operably linked transgene encodes a polypeptide. In an embodiment, a gene expression cassette comprises the synthesized polynucleotide sequence comprising a sequence identity of at least 90% to SEQ ID NO:1 operably linked to the transgene, that is operably linked to a 3′-untranslated region. In an embodiment the 3′-untranslated region comprises a polynucleotide that has a sequence identity of at least 90% to SEQ ID NO:3 or SEQ ID NO:4. In yet another embodiment, the transgene is selected from the group consisting of insecticidal resistance transgene, herbicide tolerance transgene, nitrogen use efficiency transgene, water use efficiency transgene, nutritional quality transgene, DNA binding transgene, and selectable marker transgene. In an embodiment, a recombinant vector comprises the gene expression cassette. In an additional embodiment, the recombinant vector is selected from the group consisting of a plasmid vector, a cosmid vector, and a BAC vector. In an embodiment, a transgenic cell comprises the gene expression cassette. In a further embodiment, the transgenic cell is a transgenic plant cell. In an embodiment, a transgenic plant comprises the transgenic plant cell. In a further embodiment, the transgenic plant is a monocotyledonous plant. In other embodiments, the monocotyledonous plant is selected from the group consisting of a maize plant, a wheat plant, and a rice plant. In an embodiment, a transgenic seed is obtained from the transgenic plant. In other embodiments, the synthetic polynucleotide comprises a polynucleotide sequence of nucleotides 1-2,089 of SEQ ID NO:1.

In an embodiment, a construct includes a gene expression cassette comprising a Zea mays GRMZM2G015295 gene promoter of SEQ ID NO:1 or SEQ ID NO:2. In an embodiment, a gene expression cassette includes a Zea mays GRMZM2G015295 gene promoter of SEQ ID NO:1 or SEQ ID NO:2 operably linked to a transgene or a heterologous coding sequence. In an embodiment, a gene expression cassette includes a Zea mays GRMZM2G015295 gene 3′ untranslated region of SEQ ID NO:3 or SEQ ID NO:4 operably linked to a transgene. In an embodiment, a gene expression cassette includes a Zea mays GRMZM2G015295 gene 3′ untranslated region of SEQ ID NO:3 or SEQ ID NO:4 operably linked to a promoter. In a further embodiment, a gene expression cassette includes a Zea mays GRMZM2G015295 gene 3′ untranslated region of SEQ ID NO:3 or SEQ ID NO:4 operably linked to a Zea mays GRMZM2G015295 gene promoter of SEQ ID NO:1 or SEQ ID NO:2. In an embodiment, a gene expression cassette includes a Zea mays GRMZM2G015295 gene promoter of SEQ ID NO:1 or SEQ ID NO:2 operably linked to a transgene or a heterologous coding sequence. In an embodiment, a gene expression cassette includes at least one, two, three, four, five, six, seven, eight, nine, ten, or more transgenes.

In an embodiment, a gene expression cassette includes independently a) a Zea mays GRMZM2G015295 gene promoter of SEQ ID NO:1 or SEQ ID NO:2 and b) a Zea mays GRMZM2G015295gene 3′untranslated region of SEQ ID NO:3 or SEQ ID NO:4.

Methods of growing plants expressing a transgene using Zea mays GRMZM2G015295 gene promoter of SEQ ID NO:1 or SEQ ID NO:2 and 3′untranslated region of SEQ ID NO:3 or SEQ ID NO:4 are disclosed herein. Methods of culturing plant tissues and cells expressing a transgene using the Zea mays GRMZM2G015295 gene promoter of SEQ ID NO:1 or SEQ ID NO:2 and 3′untranslated region of SEQ ID NO:3 or SEQ ID NO:4 are also disclosed herein. In an embodiment, methods as disclosed herein include tissue-specific gene expression in plant roots.

In an embodiment, a gene expression cassette includes a promoter polynucleotide sequence of SEQ ID NO:1 or SEQ ID NO:2 that was obtained from Zea mays GRMZM2G015295 gene.

### BRIEF DESCRIPTION OF THE DRAWINGS

FIG. 1 shows a schematic flow chart displaying the process of identifying high expressing genes in maize using the bioinformatics approach.

FIG. 2 shows the sequence comparison of two promoters present in pDAB113023 (2089 nt) and pDAB122815 (856 nt).

FIG. 3 shows the sequence comparison of two 3′ UTR sequences present in pDAB113023 (1049 bp) and pDAB122815 (471 bp).

FIG. 4 shows the vector plasmid map of pDAB113023 depicting a gene expression cassette comprising the Zea mays GRMZM2G015295 gene promoter and 3′ UTR regulatory elements controlling the expression of a cry34Ab1 reporter gene.

FIG. 5 shows a map of pDAB101556 control construct containing an yfp reporter gene driven by the Zea mays ubiquitin-1 (ZmUbil) promoter v2 and Zea mays Per5 (ZmPer5) 3′ UTR v2.

### SEQUENCE LISTING

SEQ ID NO:1 is the pDAB113023 promoter sequence (2089 nt).

SEQ ID NO:2 is the truncated pDAB113023 promoter sequence with repeats removed (857 nt).

SEQ ID NO:3 is the pDAB113023 3′ UTR sequence (1049 nt).

SEQ ID NO:4 is the truncated pDAB113023 3′ UTR sequence with repeats removed (471 nt).

SEQ ID NO:5 is the native sequence of the maize gene, GRMZM2G015285, of the B73 genome.

SEQ ID NO:6 is the Cry34Ab1 v2 Forward Primer (TQ.8v6.1.F).

SEQ ID NO:7 is the Cry34Ab1 v2 Reverse Primer (TQ.8v6.1.R).

SEQ ID NO:8 is the Cry34Ab1 v2 Probe (TQ.8v6.1.MGB.P).

SEQ ID NO:9 is the Invertase Forward Primer (InvertaseF).

SEQ ID NO:10 is the Invertase Reverse Primer (InvertaseR).

SEQ ID NO:11 is the Invertase Probe (InvertaseProbe-HEX).

SEQ ID NO:12 is the AAD1 Forward Primer.

SEQ ID NO:13 is the AAD1 Reverse Primer.

SEQ ID NO:14 is the AAD1 Probe.

### DETAILED DESCRIPTION

As used herein, the articles, “a”, “an”, and “the” include plural references unless the context clearly and unambiguously dictates otherwise.

As used herein, the term “backcrossing” refers to a process in which a breeder crosses hybrid progeny back to one of the parents, for example, a first generation hybrid F1 with one of the parental genotypes of the F1 hybrid.

As used herein, the term “intron” refers to any nucleic acid sequence comprised in a gene (or expressed nucleotide sequence of interest) that is transcribed but not translated. Introns include untranslated nucleic acid sequence within an expressed sequence of DNA, as well as corresponding sequence in RNA molecules transcribed therefrom.

A construct described herein can also contain sequences that enhance translation and/or mRNA stability such as introns. An example of one such intron is the first intron of gene II of the histone variant of Arabidopsis thaliana or any other commonly known intron sequence. Introns can be used in combination with a promoter sequence to enhance translation and/or mRNA stability.

As used herein, the term “3′ untranslated region” or “3′-UTR” refers to an untranslated segment in a 3′ terminus of the pre-mRNAs or mature mRNAs. For example, on mature mRNAs this region harbors the poly-(A) tail and is known to have many roles in mRNA stability, translation initiation, and mRNA export.

As used herein, the term “polyadenylation signal” refers to a nucleic acid sequence present in mRNA transcripts that allows for transcripts, when in the presence of a poly-(A) polymerase, to be polyadenylated on the polyadenylation site, for example, located 10 to 30 bases downstream of the poly-(A) signal. Many polyadenylation signals are known in the art and are useful for the present invention. An exemplary sequence includes AAUAAA and variants thereof, as described in Loke J., et al., (2005) Plant Physiology 138(3); 1457-1468.

As used herein, the term “isolated” refers to a biological component (including a nucleic acid or protein) that has been separated from other biological components in the cell of the organism in which the component naturally occurs (i.e., other chromosomal and extra-chromosomal DNA).

As used herein, the term “purified” in reference to nucleic acid molecules does not require absolute purity (such as a homogeneous preparation); instead, it represents an indication that the sequence is relatively more pure than in its native cellular environment (compared to the natural level this level should be at least 2-5 fold greater, e.g., in terms of concentration or gene expression levels). The DNA molecules obtained directly from total DNA or from total RNA. In addition, cDNA clones are not naturally occurring, but rather are preferably obtained via manipulation of a partially purified, naturally occurring substance (messenger RNA). The construction of a cDNA library from mRNA involves the creation of a synthetic substance (cDNA). Individual cDNA clones can be purified from the synthetic library by clonal selection of the cells carrying the cDNA library. Thus, the process which includes the construction of a cDNA library from mRNA and purification of distinct cDNA clones yields an approximately 106-fold purification of the native message. Likewise, a promoter DNA sequence could be cloned into a plasmid. Such a clone is not naturally occurring, but rather is preferably obtained via manipulation of a partially purified, naturally occurring substance such as a genomic DNA library. Thus, purification of at least one order of magnitude, preferably two or three orders, and more preferably four or five orders of magnitude is favored in these techniques.

Similarly, purification represents an indication that a chemical or functional change in the component DNA sequence has occurred. Nucleic acid molecules and proteins that have been “purified” include nucleic acid molecules and proteins purified by standard purification methods. The term “purified” also embraces nucleic acids and proteins prepared by recombinant DNA methods in a host cell (e.g., plant cells), as well as chemically-synthesized nucleic acid molecules, proteins, and peptides.

The term “recombinant” means a cell or organism in which genetic recombination has occurred. It also includes a molecule (e.g., a vector, plasmid, nucleic acid, polypeptide, or a small RNA) that has been artificially or synthetically (i.e., non-naturally) altered by human intervention. The alteration can be performed on the molecule within, or removed from, its natural environment or state.

As used herein, the term “expression” refers to the process by which a polynucleotide is transcribed into mRNA (including small RNA molecules) and/or the process by which the transcribed mRNA (also referred to as “transcript”) is subsequently translated into peptides, polypeptides, or proteins. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein, the terms “homology-based gene silencing” or “HBGS” are generic terms that include both transcriptional gene silencing and post-transcriptional gene silencing. Silencing of a target locus by an unlinked silencing locus can result from transcription inhibition (transcriptional gene silencing; TGS) or mRNA degradation (post-transcriptional gene silencing; PTGS), owing to the production of double-stranded RNA (dsRNA) corresponding to promoter or transcribed sequences, respectively. Involvement of distinct cellular components in each process suggests that dsRNA-induced TGS and PTGS likely result from the diversification of an ancient common mechanism. However, a strict comparison of TGS and PTGS has been difficult to achieve because it generally relies on the analysis of distinct silencing loci. A single transgene locus can be described to trigger both TGS and PTGS, owing to the production of dsRNA corresponding to promoter and transcribed sequences of different target genes.

As used herein, the terms “nucleic acid molecule”, “nucleic acid”, or “polynucleotide” (all three terms are synonymous with one another) refer to a polymeric form of nucleotides, which may include both sense and anti-sense strands of RNA, cDNA, genomic DNA, and synthetic forms, and mixed polymers thereof. “A nucleotide” may refer to a ribonucleotide, deoxyribonucleotide, or a modified form of either type of nucleotide. A nucleic acid molecule is usually at least 10 bases in length, unless otherwise specified. The terms may refer to a molecule of RNA or DNA of indeterminate length. The terms include single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” relative to a further element if they are bonded or would be bonded to the same nucleic acid in the 5′ direction from that element. Similarly, discrete elements may be “downstream” relative to a further element if they are or would be bonded to the same nucleic acid in the 3′ direction from that element.

As used herein, the term “base position” refers to the location of a given base or nucleotide residue within a designated nucleic acid. A designated nucleic acid may be defined by alignment with a reference nucleic acid.

As used herein, the term “hybridization” refers to a process where oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and bonding of a pyrimidine to a purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

As used herein, the terms “specifically hybridizable” and “specifically complementary” refers to a sufficient degree of complementarity such that stable and specific binding occurs between an oligonucleotide and the DNA or RNA target. Oligonucleotides need not be 100% complementary to the target sequence to specifically hybridize. An oligonucleotide is specifically hybridizable when binding of the oligonucleotide to the target DNA or RNA molecule interferes with the normal function of the target DNA or RNA, and there is sufficient degree of complementarity to avoid non-specific binding of an oligonucleotide to non-target sequences under conditions where specific binding is desired, for example under physiological conditions in the case of in vivo assays or systems. Such binding is referred to as specific hybridization. Hybridization conditions resulting in particular degrees of stringency will vary depending upon the nature of the chosen hybridization method and the composition and length of the hybridizing nucleic acid sequences. Generally, the temperature of hybridization and the ionic strength (especially Na+ and/or Mg2+ concentration) of a hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, N.Y., 1989.

As used herein, the term “stringent conditions” encompasses conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes. The following are representative, non-limiting hybridization conditions:
Very High Stringency: hybridization in 5×SSC buffer at 65° C. for 16 hours; wash twice in 2×SSC buffer at room temperature for 15 minutes each; and wash twice in 0.5×SSC buffer at 65° C. for 20 minutes each.
High Stringency: Hybridization in 5-6×SSC buffer at 65-70° C. for 16-20 hours; wash twice in 2×SSC buffer at room temperature for 5-20 minutes each; and wash twice in 1×SSC buffer at 55-70° C. for 30 minutes each.
Moderate Stringency: Hybridization in 6×SSC buffer at room temperature to 55° C. for 16-20 hours; wash at least twice in 2-3×SSC buffer at room temperature to 55° C. for 20-30 minutes each.

In an embodiment, specifically hybridizable nucleic acid molecules can remain bound under very high stringency hybridization conditions. In an embodiment, specifically hybridizable nucleic acid molecules can remain bound under high stringency hybridization conditions. In an embodiment, specifically hybridizable nucleic acid molecules can remain bound under moderate stringency hybridization conditions.

As used herein, the term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in polymerase chain reaction, a technique for the amplification of small DNA sequences. In polymerase chain reaction, an oligonucleotide is typically referred to as a “primer” which allows a DNA polymerase to extend the oligonucleotide and replicate the complementary strand.

As used herein, the terms “Polymerase Chain Reaction” or “PCR” refer to a procedure or technique in which minute amounts of nucleic acid, RNA and/or DNA, are amplified as described in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, N Y, 1989).

As used herein, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, that may include constituents which are co-factors or which affect conditions such as pH and the like at various suitable temperatures. A primer is preferably a single strand sequence, such that amplification efficiency is optimized, but double stranded sequences can be utilized.

As used herein, the term “probe” refers to an oligonucleotide or polynucleotide sequence that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides.

In the Southern blot assay procedure, the probe hybridizes to a DNA fragment that is attached to a membrane. A probe includes about ten nucleotides, about 100 nucleotides, about 250 nucleotides, about 500 nucleotides, about 1,000 nucleotides, about 2,500 nucleotides, or about 5,000 nucleotides. In some embodiments, a probe includes from about 500 nucleotides to about 2,500 nucleotides.

A probe can further include a detectable label, e.g., a radioactive label, a biotinylated label, a fluorophore (Texas-Red®, fluorescein isothiocyanate, etc.). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

As used herein, the terms “sequence identity” or “identity” can be used interchangeably and refer to nucleic acid residues in two sequences that are the same when aligned for maximum correspondence over a specified comparison window.

As used herein, the term “percentage of sequence identity” refers to a value determined by comparing two optimally aligned sequences (e.g., nucleic acid sequences or amino acid sequences) over a comparison window, wherein the portion of a sequence in the comparison window may comprise additions or deletions (i.e., gaps) as compared to a reference sequence (that does not comprise additions or deletions) for optimal alignment of the two sequences. A percentage is calculated by determining the number of positions at which an identical nucleic acid or amino acid residue occurs in both sequences to yield the number of matched positions, dividing the number of matched positions by the total number of positions in the comparison window, and multiplying the result by 100 to yield the percentage of sequence identity. Methods for aligning sequences for comparison are well known. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50.

The National Center for Biotechnology Information (NCBI) Basic Local Alignment Search Tool (BLAST™; Altschul et al. (1990) J. Mol. Biol. 215:403-10) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

As used herein, the term “operably linked” refers to a nucleic acid placed into a functional relationship with another nucleic acid. Generally, “operably linked” can mean that linked nucleic acids are contiguous. Linking can be accomplished by ligation at convenient restriction sites. If such sites do not exist, synthetic oligonucleotide adaptors or linkers are ligated or annealed to the nucleic acid and used to link the contiguous polynucleotide fragment. However, elements need not be contiguous to be operably linked.

As used herein, the term “promoter” refers to a region of DNA that generally is located upstream (towards the 5′ region of a gene) of a gene and is needed to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, upstream promoters, 5′-UTR, introns, and leader sequences.

As used herein, the term “upstream-promoter” refers to a contiguous polynucleotide sequence that is sufficient to direct initiation of transcription. As used herein, an upstream-promoter encompasses the site of initiation of transcription with several sequence motifs, which include TATA Box, initiator sequence, TFIIB recognition elements and other promoter motifs (Jennifer, E. F. et al, (2002) Genes & Dev., 16: 2583-2592). The upstream promoter provides the site of action to RNA polymerase II which is a multi-subunit enzyme with the basal or general transcription factors like, TFIIA, B, D, E, F and H. These factors assemble into a transcription pre initiation complex that catalyzes the synthesis of RNA from DNA template.

The activation of the upstream-promoter is done by the additional sequence of regulatory DNA sequence elements to which various proteins bind and subsequently interact with the transcription initiation complex to activate gene expression. These gene regulatory elements sequences interact with specific DNA-binding factors. These sequence motifs may sometimes be referred to as cis-elements. Such cis-elements, to which tissue-specific or development-specific transcription factors bind, individually or in combination, may determine the spatiotemporal expression pattern of a promoter at the transcriptional level. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al. (1989) Proc. Natl. Acad. Sci. USA 86:3219-23. These cis-elements are located at a varying distance from transcription start point, some cis-elements (called proximal elements) are adjacent to a minimal core promoter region while other elements can be positioned several kilobases upstream or downstream of the promoter (enhancers).

A “DNA binding transgene” is a polynucleotide coding sequence that encodes a DNA binding protein. The DNA binding protein is subsequently able to bind to another molecule. A binding protein can bind to, for example, a DNA molecule (a DNA-binding protein), a RNA molecule (an RNA-binding protein), and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

Examples of DNA binding proteins include; meganucleases, zinc fingers, CRISPRs, and TALE binding domains that can be “engineered” to bind to a predetermined nucleotide sequence. Typically, the engineered DNA binding proteins (e.g., zinc fingers, CRISPRs, or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP, CRISPR, and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication Nos. 20110301073, 20110239315 and 20119145940.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database which stores information of existing ZFP designs and binding data. See, for example, U.S. Pat. Nos. 6,140,081; 6,453,242; 6,534,261 and 6,794,136; see also WO 98/53058; WO 98/53059; WO 98/53060; WO 02/016536 and WO 03/016496.

In other examples, the DNA-binding domain of one or more of the nucleases comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than different effector proteins into the plant cell. Among these injected proteins are transcription activator-like (TALEN) effectors which mimic plant transcriptional activators and manipulate the plant transcriptome (see Kay et al (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al (1989) Mol Gen Genet 218: 127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar strain GMI1000 and in the biovar 4 strain RS1000 (See Heuer et al (2007) Appl and Enviro Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Patent Publication No. 20110301073, incorporated by reference in its entirety.

Specificity of these TAL effectors depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues at positions 12 and 13 with the identity of the contiguous nucleotides in the TAL-effector's target sequence (see Moscou and Bogdanove, (2009) Science 326:1501 and Boch et al (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN) exhibiting activity in a yeast reporter assay (plasmid based target).

The CRISPR (Clustered Regularly Interspaced Short Palindromic Repeats)/Cas (CRISPR Associated) nuclease system is a recently engineered nuclease system based on a bacterial system that can be used for genome engineering. It is based on part of the adaptive immune response of many bacteria and Archaea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer”. Cas9 cleaves the DNA to generate blunt ends at the double-stranded break (DSB) at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al (2012) Science 337, p. 816-821, Jinek et al, (2013), eLife 2:e00471, and David Segal, (2013) eLife 2:e00563). Thus, the CRISPR/Cas system can be engineered to create a DSB at a desired target in a genome, and repair of the DSB can be influenced by the use of repair inhibitors to cause an increase in error prone repair.

In other examples, the DNA binding transgene is a site specific nuclease that comprises an engineered (non-naturally occurring) Meganuclease (also described as a homing endonuclease). The recognition sequences of homing endonucleases or meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-30 3388; Dujon et al. (1989) Gene 82:115-118; Perler et al. (1994) Nucleic Acids Res. 22, 11127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 5 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

As used herein, the term “transformation” encompasses all techniques that a nucleic acid molecule can be introduced into such a cell. Examples include, but are not limited to: transfection with viral vectors; transformation with plasmid vectors; electroporation; lipofection; microinjection (Mueller et al. (1978) Cell 15:579-85); Agrobacterium-mediated transfer; direct DNA uptake; WHISKERS™-mediated transformation; and microprojectile bombardment. These techniques may be used for both stable transformation and transient transformation of a plant cell. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” refers to the introduction of a nucleic acid fragment into the nucleus, or DNA-containing organelle, of a host organism resulting in gene expression without genetically stable inheritance.

As used herein, the term “transduce” refers to a process where a virus transfers nucleic acid into a cell.

As used herein, the term “transgene” refers to an exogenous nucleic acid sequence. In one example, a transgene is a gene sequence (e.g., an herbicide-tolerance gene), a gene encoding an industrially or pharmaceutically useful compound, or a gene encoding a desirable agricultural trait. In yet another example, a transgene is a small RNA, such as an antisense nucleic acid sequence, wherein expression of the small RNA sequence inhibits expression of a target nucleic acid sequence. A transgene may contain regulatory sequences operably linked to the transgene (e.g., a promoter, intron, or 3′-UTR). In some embodiments, a nucleic acid of interest is a transgene. However, in other embodiments, a nucleic acid of interest is an endogenous nucleic acid, wherein additional genomic copies of the endogenous nucleic acid are desired, or a nucleic acid that is in the antisense orientation with respect to the sequence of a target nucleic acid in a host organism.

As used herein, the term “small RNA” refers to several classes of non-coding ribonucleic acid (ncRNA). The term small RNA describes the short chains of ncRNA produced in bacterial cells, animals, plants, and fungi. These short chains of ncRNA may be produced naturally within the cell or may be produced by the introduction of an exogenous sequence that expresses the short chain or ncRNA. The small RNA sequences do not directly code for a protein, and differ in function from other RNA in that small RNA sequences are only transcribed and not translated. The small RNA sequences are involved in other cellular functions, including gene expression and modification. Small RNA molecules are usually made up of about 20 to 30 nucleotides. The small RNA sequences may be derived from longer precursors. The precursors form structures that fold back on each other in self-complementary regions; they are then processed by the nuclease Dicer in animals or DCL1 in plants.

Many types of small RNA exist either naturally or produced artificially, including microRNAs (miRNAs), short interfering RNAs (siRNAs), antisense RNA, short hairpin RNA (shRNA), and small nucleolar RNAs (snoRNAs). Certain types of small RNA, such as microRNA and siRNA, are important in gene silencing and RNA interference (RNAi). Gene silencing is a process of genetic regulation in which a gene that would normally be expressed is “turned off” by an intracellular element, in this case, the small RNA. The protein that would normally be formed by this genetic information is not formed due to interference, and the information coded in the gene is blocked from expression.

As used herein, the term “small RNA” encompasses RNA molecules described in the literature as “tiny RNA” (Storz, (2002) Science 296:1260-3; Illangasekare et al., (1999) RNA 5:1482-1489); prokaryotic “small RNA” (sRNA) (Wassarman et al., (1999) Trends Microbiol. 7:37-45); eukaryotic “noncoding RNA (ncRNA)”; “micro-RNA (miRNA)”; “small non-mRNA (snmRNA)”; “functional RNA (fRNA)”; “transfer RNA (tRNA)”; “catalytic RNA” [e.g., ribozymes, including self-acylating ribozymes (Illangaskare et al., (1999) RNA 5:1482-1489); “small nucleolar RNAs (snoRNAs)”; “tmRNA” (a.k.a. “10S RNA”, Muto et al., (1998) Trends Biochem Sci. 23:25-29; and Gillet et al., (2001) Mol Microbiol. 42:879-885); RNAi molecules including without limitation “small interfering RNA (siRNA)”, “endoribonuclease-prepared siRNA (e-siRNA)”, “short hairpin RNA (shRNA)”, and “small temporally regulated RNA (stRNA)”; “diced siRNA (d-siRNA)”, and aptamers, oligonucleotides and other synthetic nucleic acids that comprise at least one uracil base.

As used herein, the term “vector” refers to a nucleic acid molecule as introduced into a cell, thereby producing a transformed cell. A vector may include nucleic acid sequences that permit it to replicate in the host cell, such as an origin of replication. Examples include, but are not limited to, a plasmid, cosmid, bacteriophage, bacterial artificial chromosome (BAC), or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector may optionally include materials to aid in achieving entry of the nucleic acid molecule into the cell (e.g., a liposome).

As used herein, the terms “cassette”, “expression cassette”, and “gene expression cassette” refer to a segment of DNA that can be inserted into a nucleic acid or polynucleotide at specific restriction sites or by homologous recombination. A segment of DNA comprises a polynucleotide containing a gene of interest that encodes a small RNA or a polypeptide of interest, and the cassette and restriction sites are designed to ensure insertion of the cassette in the proper reading frame for transcription and translation. In an embodiment, an expression cassette can include a polynucleotide that encodes a small RNA or a polypeptide of interest and having elements in addition to the polynucleotide that facilitate transformation of a particular host cell. In an embodiment, a gene expression cassette may also include elements that allow for enhanced expression of a small RNA or a polynucleotide encoding a polypeptide of interest in a host cell. These elements may include, but are not limited to: a promoter, a minimal promoter, an enhancer, a response element, an intron, a 5′ untranslated, a 3′ untranslated region sequence, a terminator sequence, a polyadenylation sequence, and the like.

As used herein, the term “heterologous coding sequence” is used to indicate any polynucleotide that codes for, or ultimately codes for, a peptide or protein or its equivalent amino acid sequence, e.g., an enzyme, that is not normally present in the host organism and can be expressed in the host cell under proper conditions. As such, “heterologous coding sequences” may include one or additional copies of coding sequences that are not normally present in the host cell, such that the cell is expressing additional copies of a coding sequence that are not normally present in the cells. The heterologous coding sequences can be RNA or any type thereof, e.g., mRNA, DNA or any type thereof, e.g., cDNA, or a hybrid of RNA/DNA. Examples of coding sequences include, but are not limited to, full-length transcription units that comprise such features as the coding sequence, introns, promoter regions, 5′-UTR, 3′-UTRs and enhancer regions.

“Heterologous coding sequences” also includes the coding portion of the peptide or enzyme, i.e., the cDNA or mRNA sequence, of the peptide or enzyme, as well as the coding portion of the full-length transcriptional unit, i.e., the gene comprising introns and exons, as well as “codon optimized” sequences, truncated sequences or other forms of altered sequences that code for the enzyme or code for its equivalent amino acid sequence, provided that the equivalent amino acid sequence produces a functional protein. Such equivalent amino acid sequences can have a deletion of one or more amino acids, with the deletion being N-terminal, C-terminal, or internal. Truncated forms are envisioned as long as they have the catalytic capability indicated herein.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known plant exhibiting the desired expression, and a negative control, such as a sample from a known plant lacking the desired expression.

As used herein, the term “plant” includes plants and plant parts including but not limited to plant cells and plant tissues such as leaves, stems, roots, flowers, pollen, and seeds. A class of plant that can be used in the present invention is generally as broad as the class of higher and lower plants amenable to mutagenesis including angiosperms, gymnosperms, ferns, and multicellular algae. Thus, “plant” includes dicot and monocot plants. Examples of dicotyledonous plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledonous plants include corn, rice, wheat, sugarcane, barley, rye, sorghum, orchids, bamboo, banana, cattails, lilies, oat, onion, millet, and triticale.

As used herein, the term “plant material” refers to leaves, stems, roots, flowers or flower parts, fruits, pollen, egg cells, zygotes, seeds, cuttings, cell or tissue cultures, or any other part or product of a plant. In an embodiment, plant material includes cotyledon and leaf. In an embodiment, plant material includes root tissues and other plant tissues located underground.

As used herein, the term “selectable marker gene” refers to a gene that is optionally used in plant transformation to, for example, protect plant cells from a selective agent or provide resistance/tolerance to a selective agent. In addition, “selectable marker gene” is meant to encompass reporter genes. Only those cells or plants that receive a functional selectable marker are capable of dividing or growing under conditions having a selective agent. Examples of selective agents can include, for example, antibiotics, including spectinomycin, neomycin, kanamycin, paromomycin, gentamicin, and hygromycin. These selectable markers include neomycin phosphotransferase (npt II), which expresses an enzyme conferring resistance to the antibiotic kanamycin, and genes for the related antibiotics neomycin, paromomycin, gentamicin, and G418, or the gene for hygromycin phosphotransferase (hpt), which expresses an enzyme conferring resistance to hygromycin. Other selectable marker genes can include genes encoding herbicide tolerance including bar or pat (tolerance against glufosinate ammonium or phosphinothricin), acetolactate synthase (ALS, tolerance against inhibitors such as sulfonylureas (SUs), imidazolinones (IMIs), triazolopyrimidines (TPs), pyrimidinyl oxybenzoates (POBs), and sulfonylamino carbonyl triazolinones that prevent the first step in the synthesis of the branched-chain amino acids), glyphosate, 2,4-D, and metal resistance or sensitivity. Examples of “reporter genes” that can be used as a selectable marker gene include the visual observation of expressed reporter gene proteins such as proteins encoding β-glucuronidase (GUS), luciferase, green fluorescent protein (GFP), yellow fluorescent protein (YFP), DsRed, β-galactosidase, chloramphenicol acetyltransferase (CAT), alkaline phosphatase, and the like. The phrase “marker-positive” refers to plants that have been transformed to include a selectable marker gene.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker can be attached by spacer arms of various lengths to reduce potential steric hindrance.

As used herein, the term “detecting” is used in the broadest sense to include both qualitative and quantitative measurements of a specific molecule, for example, measurements of a specific polypeptide.

Unless otherwise specifically explained, all technical and scientific terms used herein have the same meaning as commonly understood by those of ordinary skill in the art that this disclosure belongs. Definitions of common terms in molecular biology can be found in, for example: Lewin, Genes V, Oxford University Press, 1994; Kendrew et al. (eds.), The Encyclopedia of Molecular Biology, Blackwell Science Ltd., 1994; and Meyers (ed.), Molecular Biology and Biotechnology: A Comprehensive Desk Reference, VCH Publishers, Inc., 1995.

Regulatory Elements

Plant promoters used for basic research or biotechnological applications are generally unidirectional, directing the expression of transgene that has been fused at its 3′ end (downstream). It is often necessary to robustly express transgenes within plants for metabolic engineering and trait stacking. In addition, multiple novel promoters are typically required in transgenic crops to drive the expression of multiple genes. Disclosed, herein is a promoter that can direct the expression of a first gene that has been fused at its 3′ end (downstream).

Development of transgenic products is becoming increasingly complex, which requires robustly expressing transgenes and stacking multiple transgenes into a single locus. Traditionally, each transgene requires a unique promoter for expression wherein multiple promoters are required to express different transgenes within one gene stack. With an increasing size of gene stacks, this frequently leads to repeated use of the same promoter to obtain similar levels of expression patterns of different transgenes for expression of a single polygenic trait. Multi-gene constructs driven by the same promoter are known to cause gene silencing resulting in less efficacious transgenic products in the field. Excess of transcription factor (TF)-binding sites due to promoter repetition can cause depletion of endogenous TFs leading to transcriptional inactivation. The silencing of transgenes will likely undesirably affect performance of a transgenic plant produced to express transgenes. Repetitive sequences within a transgene may lead to gene intra locus homologous recombination resulting in polynucleotide rearrangements.

Tissue specific (i.e., tissue-preferred) or organ specific promoters drive gene expression in a certain tissue such as in the kernel, root, leaf, or tapetum of the plant. Tissue and developmental stage specific promoters derive the expression of genes, which are expressed in particular tissues or at particular time periods during plant development. Tissue specific promoters are required for certain applications in the transgenic plants industry and are desirable as they permit specific expression of heterologous genes in a tissue and/or developmental stage selective manner, indicating expression of the heterologous gene differentially at a various organs, tissues and/or times, but not in other. For example, increased resistance of a plant to infection by soil-borne pathogens might be accomplished by transforming the plant genome with a pathogen-resistance gene such that pathogen-resistance protein is robustly expressed within the roots of the plant. Alternatively, it may be desirable to express a transgene in plant tissues that are in a particular growth or developmental phase such as, for example, cell division or elongation. Another application is the desirability of using tissue specific promoters, e.g. such that would confine the expression of the transgenes encoding an agronomic trait in developing xylem. One particular problem remaining in the identification of tissue specific promoters is how to identify the potentially most important genes and their corresponding promoters, and to relate these to specific developmental properties of the cell. Another problem is to clone all relevant cis-acting transcriptional control elements so that the cloned DNA fragment drives transcription in the wanted specific expression pattern. A particular problem is to identify tissue-specific promoters, related to specific cell types, developmental stages and/or functions in the plant that are not expressed in other plant tissues.

Provided are methods and constructs using Zea mays GRMZM2G015295 gene promoter regulatory elements to express transgenes in plant. In an embodiment, a promoter can be a Zea mays GRMZM2G015295 gene promoter of SEQ ID NO:1 or SEQ ID NO:2.

In an embodiment, a gene expression cassette comprises a promoter. In an embodiment, a promoter can be a Zea mays GRMZM2G015295 gene promoter of the subject disclosure. In an embodiment, a gene expression cassette comprises a promoter, wherein the promoter is at least 80%, 85%, 90%, 91%, 92%, 93%, 94%, 95%, 96%, 97%, 98%, 99%, 99.5%, 99.8%, or 100% identical to SEQ ID NO:1 or SEQ ID NO:2. In an embodiment, a gene expression cassette comprises a Zea mays GRMZM2G015295 gene promoter that is operably linked to a transgene. In an embodiment, a gene expression cassette comprising the Zea mays GRMZM2G015295 gene promoter may drive expression of two or more transgenes. In an illustrative embodiment, a gene expression cassette comprises a Zea mays GRMZM2G015295 gene promoter that is operably linked to a transgene, wherein the transgene can be an insecticidal resistance transgene, a herbicide tolerance transgene, a nitrogen use efficiency transgene, a water use efficiency transgene, a nutritional quality transgene, a DNA binding transgene, a selectable marker transgene, or combinations thereof.

Transgene expression may also be regulated by a 3′-untranslated gene region (i.e., 3′-UTR) located downstream of the gene's coding sequence. Both a promoter and a 3′-UTR can regulate transgene expression. While a promoter is necessary to drive transcription, a 3′-UTR gene region can terminate transcription and initiate polyadenylation of a resulting mRNA transcript for translation and protein synthesis. A 3′-UTR gene region aids stable expression of a transgene. In an embodiment, a 3′-UTR can be a Zea mays GRMZM2G015295 gene 3′-UTR of SEQ ID NO:3 or SEQ ID NO:4.

In an embodiment, a gene expression cassette comprises a 3′-UTR. In an embodiment, a 3′-UTR can be a Zea mays GRMZM2G015295 3′-UTR. In an embodiment, a gene expression cassette comprises a 3′-UTR, wherein the 3′-UTR is at least 80%, 85%, 90%, 91%, 92%, 93%, 94%, 95%, 96%, 97%, 98%, 99%, 99.5%, 99.8%, or 100% identical to SEQ ID NO:3 or SEQ ID NO:4. In an embodiment, a gene expression cassette comprises a Zea mays GRMZM2G015295 gene 3′-UTR that is operably linked to a transgene. In an illustrative embodiment, a gene expression cassette comprises a 3′-UTR that is operably linked to a transgene, wherein the transgene can be an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof.

In an embodiment, a gene expression cassette comprises a promoter and a 3′ untranslated region purified from the Zea mays GRMZM2G015295 gene. In an embodiment, a gene expression cassette comprises: a) a promoter, wherein the promoter is at least 80%, 85%, 90%, 91%, 92%, 93%, 94%, 95%, 96%, 97%, 98%, 99%, 99.5%, 99.8%, or 100% identical to SEQ ID NO:1 or SEQ ID NO:2; and/or, b) a 3′untranslated region, wherein the 3′untranslated region is at least 80%, 85%, 90%, 91%, 92%, 93%, 94%, 95%, 96%, 97%, 98%, 99%, 99.5%, 99.8%, or 100% identical to SEQ ID NO:3 or SEQ ID NO:4.

For example, a gene expression cassette may include both a promoter and a 3′-UTR wherein the promoter is a polynucleotide of SEQ ID NO:1, and the 3′-UTR is a polynucleotide of SEQ ID NO:3. In another embodiment, a gene expression cassette may include both a promoter and a 3′-UTR wherein the promoter is a polynucleotide of SEQ ID NO:1, and the 3′-UTR is a polynucleotide of SEQ ID NO:4. In a subsequent embodiment, a gene expression cassette may include both a promoter and a 3′-UTR wherein the promoter is a polynucleotide of SEQ ID NO:2, and the 3′-UTR is a polynucleotide of SEQ ID NO:3. In yet another embodiment, a gene expression cassette may include both a promoter and a 3′-UTR wherein the promoter is a polynucleotide of SEQ ID NO:2, and the 3′-UTR is a polynucleotide of SEQ ID NO:4.

A promoter and a 3′ untranslated region can be operably linked to different transgenes within a gene expression cassette when a gene expression cassette includes one or more transgenes. In an illustrative embodiment, a gene expression cassette comprises a Zea mays gene promoter (SEQ ID NO:1 or SEQ ID NO:2) that is operably linked to a transgene, wherein the transgene can be an insecticidal resistance transgene, a herbicide tolerance transgene, a nitrogen use efficiency transgene, a water use efficiency transgene, a nutritional quality transgene, a DNA binding transgene, a selectable marker transgene, or combinations thereof. In an illustrative embodiment, a gene expression cassette comprises a Zea mays gene 3′ untranslated region (SEQ ID NO:3 or SEQ ID NO:4) that is operably linked to a transgene, wherein the transgene can be an insecticidal resistance transgene, a herbicide tolerance transgene, a nitrogen use efficiency transgene, a water us efficiency transgene, a nutritional quality transgene, a DNA binding transgene, a selectable marker transgene, or combinations thereof.

In an embodiment, a vector comprises a gene expression cassette as disclosed herein. In an embodiment, a vector can be a plasmid, a cosmid, a bacterial artificial chromosome (BAC), a bacteriophage, a virus, or an excised polynucleotide fragment for use in transformation or gene targeting such as a donor DNA.

In an embodiment, a cell or plant comprises a gene expression cassette as disclosed herein. In an embodiment, a cell or plant comprises a vector comprising a gene expression cassette as disclosed herein. In an embodiment, a vector can be a plasmid, a cosmid, a bacterial artificial chromosome (BAC), a bacteriophage, or a virus. Thereby, a cell or plant comprising a gene expression cassette as disclosed herein is a transgenic cell or transgenic plant, respectively. In an embodiment, a transgenic plant can be a monocotyledonous plant. In an embodiment, a transgenic monocotyledonous plant can be, but is not limited to maize, wheat, rice, sorghum, oats, rye, bananas, sugar cane, and millet. In an embodiment, a transgenic plant can be a dicotyledonous plant. In an embodiment, a transgenic dicotyledonous plant can be, but is not limited to soybean, cotton, sunflower, and canola. An embodiment also includes a transgenic seed from a transgenic plant as disclosed herein.

In an embodiment, a gene expression cassette includes two or more transgenes. The two or more transgenes may be operably linked to a Zea mays GRMZM2G015295 gene promoter or 3′untranslated region as disclosed herein. In an embodiment, a gene expression cassette includes one or more transgenes. In an embodiment with one or more transgenes, at least one transgene is operably linked to a Zea mays GRMZM2G015295 gene promoter or 3′untranslated region of the subject disclosure.

Selectable Markers

Various selectable markers also described as reporter genes can be incorporated into a chosen expression vector to allow for identification and selection of transformed plants (“transformants”). Many methods are available to confirm expression of selectable markers in transformed plants, including for example DNA sequencing and PCR (polymerase chain reaction), Southern blotting, RNA blotting, immunological methods for detection of a protein expressed from the vector, e g., precipitated protein that mediates phosphinothricin resistance, or visual observation of other proteins such as reporter genes encoding β-glucuronidase (GUS), luciferase, green fluorescent protein (GFP), yellow fluorescent protein (YFP), DsRed, β-galactosidase, chloramphenicol acetyltransferase (CAT), alkaline phosphatase, and the like (See Sambrook, et al., Molecular Cloning: A Laboratory Manual, Third Edition, Cold Spring Harbor Press, N.Y., 2001, the content is incorporated herein by reference in its entirety).

Selectable marker genes are utilized for selection of transformed cells or tissues. Selectable marker genes include genes encoding antibiotic resistance, such as those encoding neomycin phosphotransferase II (NEO) and hygromycin phosphotransferase (HPT) as well as genes conferring tolerance to herbicidal compounds. Herbicide tolerance genes generally code for a modified target protein insensitive to the herbicide or for an enzyme that degrades or detoxifies the herbicide in the plant before it can act. For example, tolerance to glyphosate has been obtained by using genes coding for mutant target enzymes, 5-enolpyruvylshikimate-3-phosphate synthase (EPSPS). Genes and mutants for EPSPS are well known, and further described below. Tolerance to glufosinate ammonium, bromoxynil, and 2,4-dichlorophenoxyacetate (2,4-D) have been obtained by using bacterial genes encoding pat or DSM-2, a nitrilase, an aad-1 or an aad-12 gene, which detoxifies the respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate tolerance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar genes from Streptomyces species, including Streptomyces hygroscopicus and Streptomyces viridichromo genes, and pyridinoxy or phenoxy proprionic acids and cyclohexones (ACCase inhibitor-encoding genes). Exemplary genes conferring resistance to cyclohexanediones and/or aryloxyphenoxypropanoic acid (including Haloxyfop, Diclofop, Fenoxyprop, Fluazifop, Quizalofop) include genes of acetyl coenzyme A carboxylase (ACCase)-Acc1-S1, Acc1-S2, and Acc1-S3. In an embodiment, herbicides can inhibit photosynthesis, including triazine (psbA and 1s+ genes) or benzonitrile (nitrilase gene).

In an embodiment, selectable marker genes include, but are not limited to genes encoding: neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA).

An embodiment also includes genes encoding resistance to: chloramphenicol; methotrexate; hygromycin; spectinomycin; bromoxynil; glyphosate; and phosphinothricin.

The above list of selectable marker genes is not meant to be limiting. Any reporter or selectable marker gene are encompassed by the present invention.

Selectable marker genes are synthesized for optimal expression in a plant. For example, in an embodiment, a coding sequence of a gene has been modified by codon optimization to enhance expression in plants. A selectable marker gene can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. In an embodiment, a selectable marker gene is designed to be expressed in plants at a higher level resulting in higher transformation efficiency. Methods for plant optimization of genes are well known. Guidance regarding the optimization and manufacture of synthetic polynucleotide sequences can be found in, for example, WO2013016546, WO2011146524, WO1997013402, U.S. Pat. No. 6,166,302, and U.S. Pat. No. 5,380,831, herein incorporated by reference.

Transgenes

The disclosed methods and compositions can be used to express polynucleotide gene sequences within the plant genome. Accordingly, expression of genes encoding herbicide tolerance, insect resistance, nutrients, antibiotics, or therapeutic molecules can be driven by a plant promoter.

In one embodiment the Zea mays GRMZM2G015295 gene regulatory element of the subject disclosure is combined or operably linked with gene encoding polynucleotide sequences that provide resistance or tolerance to glyphosate or another herbicide, and/or provides resistance to select insects or diseases and/or nutritional enhancements, and/or improved agronomic characteristics, and/or proteins or other products useful in feed, food, industrial, pharmaceutical, or other uses. The transgenes can be “stacked” with two or more nucleic acid sequences of interest within a plant genome. Stacking can be accomplished, for example, via conventional plant breeding using two or more events, transformation of a plant with a construct which contains the sequences of interest, re-transformation of a transgenic plant, or addition of new traits through targeted integration via homologous recombination.

Such polynucleotide sequences of interest include, but are not limited to, those examples provided below:

1. Genes or Coding Sequence (e.g. iRNA) That Confer Resistance to Pests or Disease

(A) Plant Disease Resistance Genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium flavum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089).

(B) A Bacillus thuringiensis protein, a derivative thereof or a synthetic polypeptide modeled thereon, such as, a nucleotide sequence of a Bt δ-endotoxin gene (Geiser et al., 1986 Gene 48:109), and a vegetative insecticidal (VIP) gene (see, e.g., Estruch et al. (1996) Proc. Natl. Acad. Sci. 93:5389-94). Moreover, DNA molecules encoding δ-endotoxin genes can be purchased from American Type Culture Collection (Rockville, Md.), under ATCC accession numbers 40098, 67136, 31995, and 31998.

(C) A lectin, such as, nucleotide sequences of several Clivia miniata mannose-binding lectin genes (Van Damme et al., 1994 Plant Molec. Biol. 24:825).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al., 1987 J. Biol. Chem. 262:16793), a tobacco proteinase inhibitor I (Huub et al., 1993 Plant Molec. Biol. 21:985), and an a-amylase inhibitor (Sumitani et al., 1993 Biosci. Biotech. Biochem. 57:1243).

(F) An insect-specific hormone or pheromone such as an ecdysteroid and juvenile hormone, a variant thereof, a mimetic based thereon, or an antagonist or agonist thereof, such as baculovirus expression of cloned juvenile hormone esterase, an inactivator of juvenile hormone (Hammock et al., 1990 Nature 344:458).

(G) An insect-specific peptide or neuropeptide which, upon expression, disrupts the physiology of the affected pest (J. Biol. Chem. 269:9). Examples of such genes include an insect diuretic hormone receptor (Regan, 1994), an allostatin identified in Diploptera punctata (Pratt, 1989), and insect-specific, paralytic neurotoxins (U.S. Pat. No. 5,266,361).

(H) An insect-specific venom produced in nature by a snake, a wasp, etc., such as a scorpion insectotoxic peptide (Pang, 1992 Gene 116:165).

(I) An enzyme responsible for a hyperaccumulation of monoterpene, a sesquiterpene, a steroid, hydroxamic acid, a phenylpropanoid derivative or another non-protein molecule with insecticidal activity.

(J) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al., 1993 Insect Molec. Biol. 23:691), and parsley ubi4-2 polyubiquitin gene (Kawalleck et al., 1993 Plant Molec. Biol. 21:673).

(K) A molecule that stimulates signal transduction. Examples of such molecules include nucleotide sequences for mung bean calmodulin cDNA clones (Botella et al., 1994 Plant Molec. Biol. 24:757) and a nucleotide sequence of a maize calmodulin cDNA clone (Griess et al., 1994 Plant Physiol. 104:1467).

(L) A hydrophobic moment peptide. See U.S. Pat. Nos. 5,659,026 and 5,607,914; the latter teaches synthetic antimicrobial peptides that confer disease resistance.

(M) A membrane permease, a channel former or a channel blocker, such as a cecropin-β lytic peptide analog (Jaynes et al., 1993 Plant Sci. 89:43) which renders transgenic tobacco plants resistant to Pseudomonas solanacearum.

(N) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al. (1990) Ann. Rev. Phytopathol. 28:451.

(O) An insect-specific antibody or an immunotoxin derived therefrom. Thus, an antibody targeted to a critical metabolic function in the insect gut would inactivate an affected enzyme, killing the insect. For example, Taylor et al. (1994) Abstract #497, Seventh Int'l. Symposium on Molecular Plant-Microbe Interactions shows enzymatic inactivation in transgenic tobacco via production of single-chain antibody fragments.

(P) A virus-specific antibody. See, for example, Tavladoraki et al. (1993) Nature 266:469, which shows that transgenic plants expressing recombinant antibody genes are protected from virus attack.

(Q) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al., 1992 Bio/Technology 10:1436). The cloning and characterization of a gene which encodes a bean endopolygalacturonase-inhibiting protein is described by Toubart et al. (1992 Plant J. 2:367).

(R) A developmental-arrestive protein produced in nature by a plant, such as the barley ribosome-inactivating gene that provides an increased resistance to fungal disease (Longemann et al., 1992 Bio/Technology 10:3305).

(S) RNA interference, in which an RNA molecule is used to inhibit expression of a target gene. An RNA molecule in one example is partially or fully double stranded, which triggers a silencing response, resulting in cleavage of dsRNA into small interfering RNAs, which are then incorporated into a targeting complex that destroys homologous mRNAs. See, e.g., Fire et al., U.S. Pat. No. 6,506,559; Graham et al. U.S. Pat. No. 6,573,099.

2. Genes That Confer Tolerance to a Herbicide

(A) Genes encoding resistance or tolerance to a herbicide that inhibits the growing point or meristem, such as an imidazalinone, sulfonanilide, or sulfonylurea herbicides. Exemplary genes in this category code for mutant acetolactate synthase (ALS) (Lee et al., 1988 EMBOJ. 7:1241) also known as acetohydroxyacid synthase (AHAS) enzyme (Miki et al., 1990 Theor. Appl. Genet. 80:449).

(B) One or more additional genes encoding resistance or tolerance to glyphosate imparted by mutant EPSP synthase and aroA genes, or through metabolic inactivation by genes such as DGT-28, 2mEPSPS, GAT (glyphosate acetyltransferase) or GOX (glyphosate oxidase) and other phosphono compounds such as glufosinate (pat,bar, and dsm-2 genes), and aryloxyphenoxypropionic acids and cyclohexanediones (ACCase inhibitor encoding genes). See, for example, U.S. Pat. No. 4,940,835, which discloses the nucleotide sequence of a form of EPSP which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061. European patent application No. 0 333 033 and U.S. Pat. No. 4,975,374 discloses nucleotide sequences of glutamine synthetase genes which confer resistance to herbicides such as L-phosphinothricin. The nucleotide sequence of a phosphinothricinacetyl-transferase gene is provided in European application No. 0 242 246. De Greef et al. (1989 Bio/Technology 7:61) describe the production of transgenic plants that express chimeric bar genes coding for phosphinothricin acetyl transferase activity. Exemplary of genes conferring resistance to aryloxyphenoxypropionic acids and cyclohexanediones, such as sethoxydim and haloxyfop, are the Acc1-S1, Acc1-S2 and Acc1-S3 genes described by Marshall et al. (1992 Theor. Appl. Genet. 83:435).

(C) Genes encoding resistance or tolerance to a herbicide that inhibits photosynthesis, such as a triazine (psbA and gs+ genes) and a benzonitrile (nitrilase gene). Przibilla et al. (1991 Plant Cell 3:169) describe the use of plasmids encoding mutant psbA genes to transform Chlamydomonas. Nucleotide sequences for nitrilase genes are disclosed in U.S. Pat. No. 4,810,648, and DNA molecules containing these genes are available under ATCC accession numbers 53435, 67441 and 67442. Cloning and expression of DNA coding for a glutathione 5-transferase is described by Hayes et al. (1992 Biochem. J. 285:173).

(D) Genes encoding resistance or tolerance to a herbicide that bind to hydroxyphenylpyruvate dioxygenases (HPPD), enzymes which catalyze the reaction in which para-hydroxyphenylpyruvate (HPP) is transformed into homogentisate. This includes herbicides such as isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular 2-cyano-3-cyclopropyl-1-(2-SO2CH3-4-CF3 phenyl)propane-1,3-dione and 2-cyano-3-cyclopropyl-1-(2-S02CH3-4-2,3Cl2phenyl)propane-1,3-dione, triketones (EP625505, EP625508, U.S. Pat. No. 5,506,195), in particular sulcotrione, and pyrazolinates. A gene that produces an overabundance of HPPD in plants can provide tolerance or resistance to such herbicides, including, for example, genes described in U.S. Pat. Nos. 6,268,549 and 6,245,968 and U.S. Patent Application, Publication No. 20030066102.

(E) Genes encoding resistance or tolerance to phenoxy auxin herbicides, such as 2,4-dichlorophenoxyacetic acid (2,4-D) and which may also confer resistance or tolerance to aryloxyphenoxypropionate (AOPP) herbicides. Examples of such genes include the α-ketoglutarate-dependent dioxygenase enzyme (aad-1) gene, described in U.S. Pat. No. 7,838,733.

(F) Genes encoding resistance or tolerance to phenoxy auxin herbicides, such as 2,4-dichlorophenoxyacetic acid (2,4-D) and which may also confer resistance or tolerance to pyridyloxy auxin herbicides, such as fluroxypyr or triclopyr. Examples of such genes include the α-ketoglutarate-dependent dioxygenase enzyme gene (aad-12), described in WO 2007/053482 A2.

(G) Genes encoding resistance or tolerance to dicamba (see, e.g., U.S. Patent Publication No. 20030135879).

(H) Genes providing resistance or tolerance to herbicides that inhibit protoporphyrinogen oxidase (PPO) (see U.S. Pat. No. 5,767,373).

(I) Genes providing resistance or tolerance to triazine herbicides (such as atrazine) and urea derivatives (such as diuron) herbicides which bind to core proteins of photosystem II reaction centers (PS II) (See Brussian et al., (1989) EMBO J. 1989, 8(4): 1237-1245).

3. Genes That Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al., 1992 Proc. Nat. Acad. Sci. USA 89:2624).

(B) Decreased phytate content

(1) Introduction of a phytase-encoding gene, such as the Aspergillus niger phytase gene (Van Hartingsveldt et al., 1993 Gene 127:87), enhances breakdown of phytate, adding more free phosphate to the transformed plant.

(2) A gene could be introduced that reduces phytate content. In maize, this, for example, could be accomplished by cloning and then reintroducing DNA associated with the single allele which is responsible for maize mutants characterized by low levels of phytic acid (Raboy et al., 1990 Maydica 35:383).

(C) Modified carbohydrate composition effected, for example, by transforming plants with a gene coding for an enzyme that alters the branching pattern of starch. Examples of such enzymes include, Streptococcus pneumoniae fructosyltransferase gene (Shiroza et al., 1988 J. Bacteriol. 170:810), Bacillus subtilis levansucrase gene (Steinmetz et al., 1985 Mol. Gen. Genel. 200:220), Bacillus licheniformis a-amylase (Pen et al., 1992 Bio/Technology 10:292), tomato invertase genes (Elliot et al., 1993), barley amylase gene (Sogaard et al., 1993 J. Biol. Chem. 268:22480), and maize endosperm starch branching enzyme II (Fisher et al., 1993 Plant Physiol. 102:10450).

Transformation

Suitable methods for transformation of plants include any method that DNA can be introduced into a cell, for example and without limitation: electroporation (see, e.g., U.S. Pat. No. 5,384,253); micro-projectile bombardment (see, e.g., U.S. Pat. Nos. 5,015,580; 5,550,318; 5,538,880; 6,160,208; 6,399,861; and 6,403,865); Agrobacterium-mediated transformation (see, e.g., U.S. Pat. Nos. 5,635,055; 5,824,877; 5,591,616; 5,981,840; and 6,384,301); and protoplast transformation (see, e.g., U.S. Pat. No. 5,508,184). These methods may be used to stably transform or transiently transform a plant.

A DNA construct may be introduced directly into the genomic DNA of the plant cell using techniques such as agitation with silicon carbide fibers (See, e.g., U.S. Pat. Nos. 5,302,523 and 5,464,765), or the DNA constructs can be introduced directly to plant tissue using biolistic methods, such as DNA particle bombardment (see, e.g., Klein et al., (1987) Nature 327:70-73). Alternatively, the DNA construct can be introduced into the plant cell via nanoparticle transformation (see, e.g., U.S. Patent Publication No. 2009/0104700, incorporated herein by reference in its entirety).

In addition, gene transfer may be achieved using non-Agrobacterium bacteria or viruses such as Rhizobium sp. NGR234, Sinorhizoboium meliloti, Mesorhizobium loti, potato virus X, cauliflower mosaic virus and cassava vein mosaic virus and/or tobacco mosaic virus, see, e.g., Chung et al. (2006) Trends Plant Sci. 11(1):1-4.

Through the application of transformation techniques, cells of virtually any plant species may be stably transformed, and these cells may be developed into transgenic plants by well-known techniques. For example, techniques that may be particularly useful in the context of cotton transformation are described in U.S. Pat. Nos. 5,846,797; 5,159,135; 5,004,863; and 6,624,344; techniques for transforming Brassica plants in particular are described, for example, in U.S. Pat. No. 5,750,871; techniques for transforming soybean are described, for example, in U.S. Pat. No. 6,384,301; and techniques for transforming maize are described, for example, in U.S. Pat. Nos. 7,060,876 and 5,591,616, and International PCT Publication WO 95/06722.

After effecting delivery of an exogenous nucleic acid to a recipient cell, a transformed cell is generally identified for further culturing and plant regeneration. In order to improve the ability to identify transformants, one may desire to employ a selectable marker gene with the transformation vector used to generate the transformant. In an illustrative embodiment, a transformed cell population can be assayed by exposing the cells to a selective agent or agents, or the cells can be screened for the desired marker gene trait.

Cells that survive exposure to a selective agent, or cells that have been scored positive in a screening assay, may be cultured in media that supports regeneration of plants. In an embodiment, any suitable plant tissue culture media may be modified by including further substances, such as growth regulators. Tissue may be maintained on a basic media with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturity.

To confirm the presence of a desired nucleic acid comprising constructs provided in regenerating plants, a variety of assays may be performed. Such assays may include: molecular biological assays, such as Southern and Northern blotting and PCR; biochemical assays, such as detecting the presence of a protein product, e.g., by immunological means (ELISA, western blots, and/or LC-MS MS spectrophotometry), or by enzymatic function; plant part assays, such as leaf or root assays; and/or analysis of the phenotype of the whole regenerated plant.

Transgenic events may be screened, for example, by PCR amplification using, e.g., oligonucleotide primers specific for nucleic acid molecules of interest. PCR genotyping is understood to include, but not be limited to, polymerase-chain reaction (PCR) amplification of genomic DNA derived from isolated host plant callus tissue predicted to contain a nucleic acid molecule of interest integrated into the genome, followed by standard cloning and sequence analysis of PCR amplification products. Methods of PCR genotyping have been well described (see, e.g., Rios et al. (2002) Plant J. 32:243-53), and may be applied to genomic DNA derived from any plant species or tissue type, including cell cultures. Combinations of oligonucleotide primers that bind to both target sequence and introduced sequence may be used sequentially or multiplexed in PCR amplification reactions. Oligonucleotide primers designed to anneal to the target site, introduced nucleic acid sequences, and/or combinations of the two may be produced. Thus, PCR genotyping strategies may include, for example and without limitation: amplification of specific sequences in the plant genome; amplification of multiple specific sequences in the plant genome; amplification of non-specific sequences in the plant genome; and combinations of any of the foregoing. One skilled in the art may devise additional combinations of primers and amplification reactions to interrogate the genome. For example, a set of forward and reverse oligonucleotide primers may be designed to anneal to nucleic acid sequence(s) specific for the target outside the boundaries of the introduced nucleic acid sequence.

Forward and reverse oligonucleotide primers may be designed to anneal specifically to an introduced nucleic acid molecule, for example, at a sequence corresponding to a coding region within a nucleotide sequence of interest comprised therein, or other parts of the nucleic acid molecule. Primers may be used in conjunction with primers described herein. Oligonucleotide primers may be synthesized according to a desired sequence and are commercially available (e.g., from Integrated DNA Technologies, Inc., Coralville, Iowa). Amplification may be followed by cloning and sequencing, or by direct sequence analysis of amplification products. In an embodiment, oligonucleotide primers specific for the gene target are employed in PCR amplifications.

Method of Expressing a Transgene

In an embodiment, a method of expressing at least one transgene in a plant comprising growing a plant comprising a Zea mays GRMZM2G015295 gene promoter operably linked to at least one transgene. In an embodiment, a method of expressing at least one transgene in a plant comprising growing a plant comprising a Zea mays GRMZM2G015295 gene 3′untranslated region operably linked to at least one transgene. In an embodiment, a method of expressing at least one transgene in a plant comprising growing a plant comprising a Zea mays GRMZM2G015295 gene promoter and a Zea mays GRMZM2G015295 gene 3′untranslated region operably linked to at least one transgene.

In an embodiment, a method of expressing at least one transgene in a plant tissue or plant cell comprising culturing a plant tissue or plant cell comprising a Zea mays GRMZM2G015295 gene promoter operably linked to at least one transgene. In an embodiment, a method of expressing at least one transgene in a plant tissue or plant cell comprising culturing a plant tissue or plant cell comprising a Zea mays GRMZM2G015295 gene 3′untranslated region operably linked to at least one transgene. In an embodiment, a method of expressing at least one transgene in a plant tissue or plant cell comprising culturing a plant tissue or plant cell comprising a Zea mays GRMZM2G015295 gene promoter and a Zea mays GRMZM2G015295 gene 3′untranslated region operably linked to at least one transgene.

In an embodiment, a method of expressing at least one transgene in a plant comprises growing a plant comprising a gene expression cassette comprising a Zea mays GRMZM2G015295 gene promoter operably linked to at least one transgene. In an embodiment, a method of expressing at least one transgene in a plant comprises growing a plant comprising a gene expression cassette comprising a Zea mays GRMZM2G015295 gene 3′untranslated region operably linked to at least one transgene. In an embodiment, a method of expressing at least one transgene in a plant comprises growing a plant comprising a gene expression cassette comprising a Zea mays GRMZM2G015295 gene promoter and a Zea mays GRMZM2G015295 gene 3′untranslated region operably linked to at least one transgene.

In an embodiment, a method of expressing at least one transgene in a plant tissue or plant cell comprises culturing a plant tissue or plant cell comprising a gene expression cassette a Zea mays GRMZM2G015295 gene promoter operably linked to at least one transgene. In an embodiment, a method of expressing at least one transgene in a plant tissue or plant cell comprises culturing a plant tissue or plant cell comprising a gene expression cassette a Zea mays GRMZM2G015295 gene 3′untranslated region operably linked to at least one transgene. In an embodiment, a method of expressing at least one transgene in a plant tissue or plant cell comprises culturing a plant tissue or plant cell comprising a gene expression cassette a Zea mays GRMZM2G015295 gene promoter and a Zea mays GRMZM2G015295 gene 3′untranslated region operably linked to at least one transgene.

In an embodiment, a plant, plant tissue, or plant cell comprises a Zea mays GRMZM2G015295 gene promoter. In an embodiment, a Zea mays GRMZM2G015295 gene promoter can be SEQ ID NO:1 or SEQ ID NO:2. In an embodiment, a plant, plant tissue, or plant cell comprises a gene expression cassette comprising a Zea mays GRMZM2G015295 gene promoter, wherein the promoter is at least 80%, 85%, 90%, 91%, 92%, 93%, 94%, 95%, 96%, 97%, 98%, 99%, 99.5%, 99.8%, or 100% identical to SEQ ID NO:1 or SEQ ID NO:2. In an embodiment, a plant, plant tissue, or plant cell comprises a gene expression cassette comprising a Zea mays GRMZM2G015295 gene promoter that is operably linked to a transgene. In an illustrative embodiment, a plant, plant tissue, or plant cell comprises a gene expression cassette comprising a Zea mays GRMZM2G015295 gene promoter that is operably linked to a transgene, wherein the transgene can be an insecticidal resistance transgene, a herbicide tolerance transgene, a nitrogen use efficiency transgene, a water use efficiency transgene, a nutritional quality transgene, a DNA binding transgene, a selectable marker transgene, or combinations thereof.

In an embodiment, a plant, plant tissue, or plant cell comprises a gene expression cassette comprising a Zea mays GRMZM2G015295 gene 3′-UTR. In an embodiment, a plant, plant tissue, or plant cell comprises a gene expression cassette comprising a Zea mays GRMZM2G015295 gene 3′-UTR. In an embodiment, the Zea mays GRMZM2G015295 gene 3′-UTR is a polynucleotide of SEQ ID NO:3 or SEQ ID NO:4. In an embodiment, a plant, plant tissue, or plant cell comprises a gene expression cassette comprising a Zea mays GRMZM2G015295 gene 3′-UTR, wherein the Zea mays GRMZM2G015295 gene 3′-UTR is at least 80%, 85%, 90%, 91%, 92%, 93%, 94%, 95%, 96%, 97%, 98%, 99%, 99.5%, 99.8%, or 100% identical to SEQ ID NO:3 or SEQ ID NO:4. In an embodiment, a gene expression cassette comprises a Zea mays GRMZM2G015295 gene 3′-UTR that is operably linked to a promoter, wherein the promoter is a Zea mays GRMZM2G015295 gene promoter, or a promoter that originates from a plant (e.g., Zea mays ubiquitin 1 promoter), a virus (e.g., Cassava vein mosaic virus promoter), or a bacteria (e.g., Agrobacterium tumefaciens delta mas).

In an embodiment, a plant, plant tissue, or plant cell comprises a gene expression cassette comprising a Zea mays GRMZM2G015295 gene 3′-UTR that is operably linked to a transgene. In an illustrative embodiment, a plant, plant tissue, or plant cell comprising a gene expression cassette comprising a Zea mays GRMZM2G015295 gene 3′-UTR that is operably linked to a transgene, wherein the transgene can be an insecticidal resistance transgene, a herbicide tolerance transgene, a nitrogen use efficiency transgene, a water use efficiency transgene, a nutritional quality transgene, a DNA binding transgene, a selectable marker transgene, or combinations thereof.

In an embodiment, a plant, plant tissue, or plant cell comprises a gene expression cassette comprising a Zea mays GRMZM2G015295 gene promoter and a Zea mays GRMZM2G015295 gene 3′-UTR that are operably linked to a transgene. The promoter and 3′-UTR can be operably linked to different transgenes within a gene expression cassette when a gene expression cassette includes two or more transgenes. In an illustrative embodiment, a gene expression cassette comprises a Zea mays GRMZM2G015295 gene promoter that is operably linked to a transgene, wherein the transgene can be an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof. In an illustrative embodiment, a gene expression cassette comprises a Zea mays GRMZM2G015295 gene 3′-UTR that is operably linked to a transgene, wherein the transgene can be an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof.

In an embodiment, transgene expression using methods described herein is expressed within a plant's root tissues. In an embodiment, transgene expression includes more than one transgene expressed in the plant's root tissues. In an embodiment, a method of growing a transgenic plant as described herein includes root-preferred transgene expression. In an embodiment, a method of expressing a transgene in a plant tissue or plant cell includes root-preferred tissues and root-preferred cells. In an embodiment, the root-preferred expression includes maize root-preferred expression.

In a further embodiment, transgene expression using methods described herein is expressed within below ground plant tissues (e.g., below ground plant tissues include root tissues). In an embodiment, transgene expression includes more than one transgene expressed in below ground plant tissues. In an embodiment, a method of growing a transgenic plant as described herein includes below ground plant tissues transgene expression. In an embodiment, a method of expressing a transgene in a plant tissue or plant cell below ground plant tissues and below ground plant cells. In an embodiment, the below ground plant tissue expression includes maize below ground plant tissue expression.

In an embodiment, a plant, plant tissue, or plant cell comprises a vector comprising a Zea mays GRMZM2G015295 gene promoter or 3′-UTR regulatory element as disclosed herein. In an embodiment, a plant, plant tissue, or plant cell comprises a vector comprising a Zea mays GRMZM2G015295 gene promoter or 3′-UTR regulatory element as disclosed herein operably linked to a transgene. In an embodiment, a plant, plant tissue, or plant cell comprises a vector comprising a gene expression cassette as disclosed herein. In an embodiment, a vector can be a plasmid, a cosmid, a bacterial artificial chromosome (BAC), a bacteriophage, or a virus fragment.

In an embodiment, a plant, plant tissue, or plant cell according to the methods disclosed herein can be monocotyledonous. The monocotyledon plant, plant tissue, or plant cell can be, but not limited to corn, rice, wheat, sugarcane, barley, rye, sorghum, orchids, bamboo, banana, cattails, lilies, oat, onion, millet, and triticale.

In an embodiment, a plant, plant tissue, or plant cell according to the methods disclosed herein can be dicotyledonous. The dicotyledon plant, plant tissue, or plant cell can be, but is not limited to rapeseed, canola, Indian mustard, Ethiopian mustard, soybean, sunflower, and cotton.

With regard to the production of genetically modified plants, methods for the genetic engineering of plants are well known in the art. For instance, numerous methods for plant transformation have been developed, including biological and physical transformation protocols for dicotyledonous plants as well as monocotyledonous plants (e.g., Goto-Fumiyuki et al., Nature Biotech 17:282-286 (1999); Mild et al., Methods in Plant Molecular Biology and Biotechnology, Glick, B. R. and Thompson, J. E. Eds., CRC Press, Inc., Boca Raton, pp. 67-88 (1993)). In addition, vectors and in vitro culture methods for plant cell or tissue transformation and regeneration of plants are available, for example, in Gruber et al., Methods in Plant Molecular Biology and Biotechnology, Glick, B. R. and Thompson, J. E. Eds., CRC Press, Inc., Boca Raton, pp. 89-119 (1993).

One of skill in the art will recognize that after the exogenous sequence is stably incorporated in transgenic plants and confirmed to be operable, it can be introduced into other plants by sexual crossing. Any of a number of standard breeding techniques can be used, depending upon the species to be crossed.

A transformed plant cell, callus, tissue, or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed cells can also be identified by screening for the activities of any visible marker genes (e.g., the yfp, gfp, β-glucuronidase, luciferase, B or C1 genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) next generation sequencing (NGS) analysis; 5) protein gel electrophoresis, western blot techniques, immunoprecipitation, or enzyme-linked immunosorbent assay (ELISA), where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, Northern blots of the RNA (e.g., mRNA) isolated from the tissues of interest. Typically, if the mRNA is present or the amount of mRNA has increased, it can be assumed that the corresponding transgene is being expressed. Other methods of measuring gene and/or encoded polypeptide activity can be used. Different types of enzymatic assays can be used, depending on the substrate used and the method of detecting the increase or decrease of a reaction product or by-product. In addition, the levels of polypeptide expressed can be measured immunochemically, i.e., ELISA, RIA, EIA and other antibody based assays well known to those of skill in the art, such as by electrophoretic detection assays (either with staining or western blotting). As one non-limiting example, the detection of the AAD-1 (aryloxyalkanoate dioxygenase; see WO 2005/107437) and PAT (phosphinothricin-N-acetyl-transferase) proteins using an ELISA assay is described in U.S. Patent Publication No. 20090093366 which is herein incorporated by reference in its entirety. The transgene may be selectively expressed in some cell types or tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed comprises the transgene or gene expression cassette. The present disclosure further encompasses the progeny, clones, cell lines or cells of the transgenic plants described above wherein said progeny, clone, cell line or cell comprise the transgene or gene construct.

While the invention has been described with reference to specific methods and embodiments, it will be appreciated that various modifications and changes may be made without departing from the invention.

### EXAMPLES

### Example 1: Identification of High Expressing Regulatory Elements

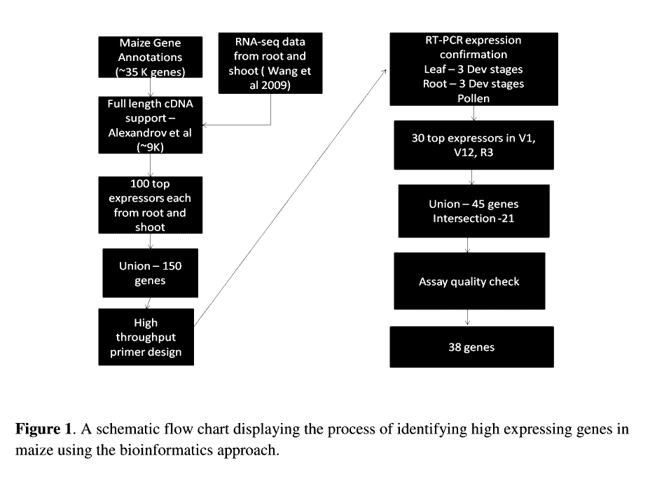

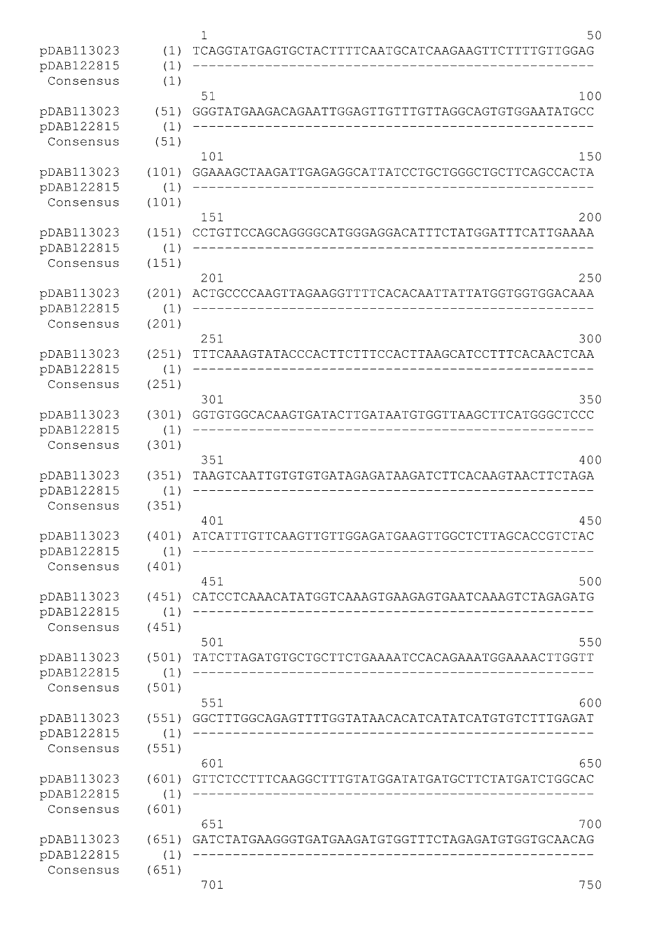

Three sources of data were considered in the study to prioritize high expressing maize genes in roots and shoots of maize seedlings: 1) 35,000 maize gene sequences and their annotations present in the public maize database in 2010 when this study was carried out; 2) gene expression data for total maize transcriptome for V4 shoots and roots (Wang, X. et al., 2009); and 3) full-length cDNA sequences of 9,000 genes (Alexandrov, N. et al., 2009). In this study, the gene expression data was aligned to both 9,000 full-length cDNA sequences and 35,000 maize genes. Based on fragments per kilobase of exon per million fragments mapped (FPKM) values, a quantitative measure of gene expression, 100 high expressing genes were identified from each of the maize root and shoot maize gene expression data by alignment to the 9,000 cDNA sequences (FIG. 1). These sequences were also mapped to the 35,000 maize genes, which aligned to the 500 best expressing genes or ˜1.4% of the best expressing genes of the maize genome.

The B73 maize genotype seed were planted in the greenhouse to collect tissue samples from 3 different stages of leaf (V4, V12 and R3), root development (V4 and V12), and from pollen for gene expression confirmation. All samples were frozen in liquid nitrogen instantly following harvesting and then stored at −80 C until use. For RNA extraction, the samples were ground with pestle and mortar in liquid nitrogen. Multiple aliquots of approximately 100 mg ground tissues were separately placed in 2 ml eppendorf tubes and the total RNA was extracted using RNeasy® Mini kit as per the manufacturer's instructions (Qiagen, Valencia, Calif.). The RNA was treated with RNase-free DNase I (Ambion, Foster City, Calif.) as per the manufacturer's instructions. The RNA was quantified using a nanodrop spectrophotometer and an aliquot was checked on a 1.2% gel to confirm RNA integrity. This RNA was employed to synthesize cDNA with a High-Capacity cDNA Reverse Transcription kit as per the manufacturer's instructions (Applied Biosystems, Foster City, Calif.). The expression of the top 150 genes in their respective tissue types was evaluated using quantitative PCR. Each PCR reaction in a final 10 μl volume contained: 5 μl Roche Syber 2× master mix, 0.10 μl 10% polyvinylpyrrolidone, 0.4 μl gene-specific forward and reverse primers from 10 μM stock,1.5 μl cDNA and 3 μl of SYBR green+water. The PCR reactions were carried out in a 96-well microtiter plate at the following cycle: pre-incubate at 95° C. for 10 min, 45 cycles of (95° C. for 0.10 min; 59° C. for 0.2 min and 72° C. for 0.2 min). The results identified the best 38 genes for leaf and root-preferred expression (FIG. 1).

### Example 2: Vector Construction

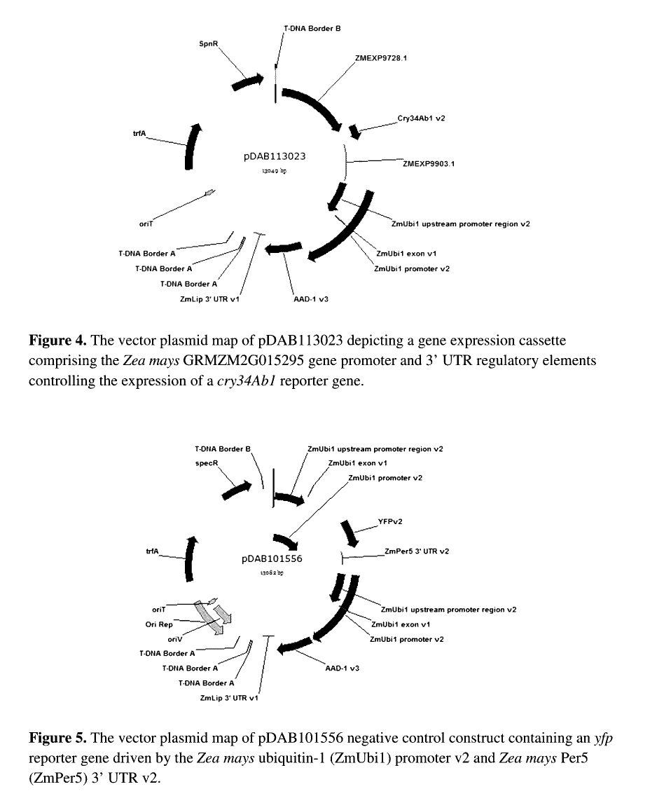

The promoter (SEQ ID NO:1) and 3′ UTR (SEQ ID NO:3) sequences were extracted from GRMZM2G015295 (SEQ ID NO:5) from the publicly available maize genome browser, because it ranked high for expression through the bioinformatics approach. The DNA elements were synthesized and cloned into entry vectors. The promoter and 3′ UTR lengths were 2089 nt and 1049 nt respectively. The promoter, cry34Ab1 reporter gene from Bacillus thurengiensis (B.t.) and the 3′ UTR were amplified with primers containing a minimum 15 nt overlapping homology to their flanking DNA element. All fragments were gel purified. All 3 fragments along with an entry vector backbone, pENTR11, were assembled in a directional order through a Geneart® Seamless cloning reaction (Invitrogen, Carlsbad, Calif.). A Gateway® LR Clonase® (Invitrogen, Carlsbad, Calif.) reaction was then performed with the resulting entry plasmid, and a destination vector, leading to a final expression vector, pDAB113023. The destination vector contained a selectable marker cassette comprised of an aad-1 gene controlled by the maize ubiquitin-1 promoter (Christensen et al., 1992) and a maize lipase 3′ UTR (U.S. Pat. No. 7,179,902) (FIG. 4).

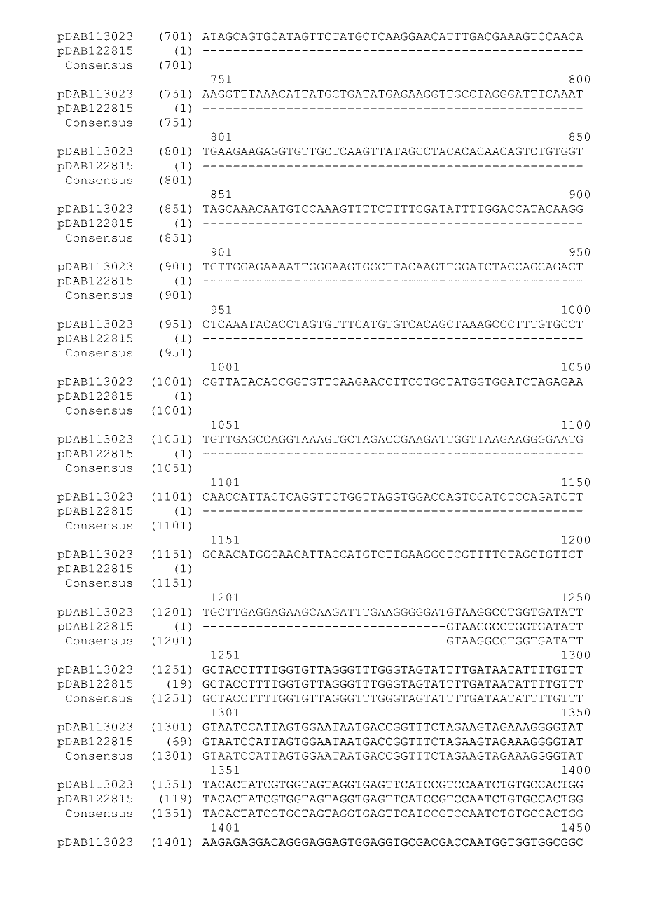

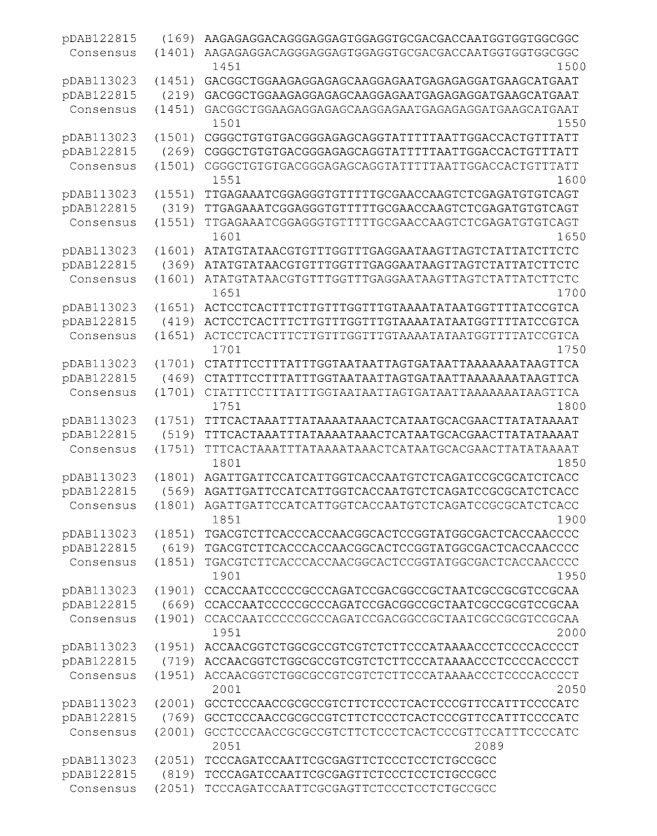

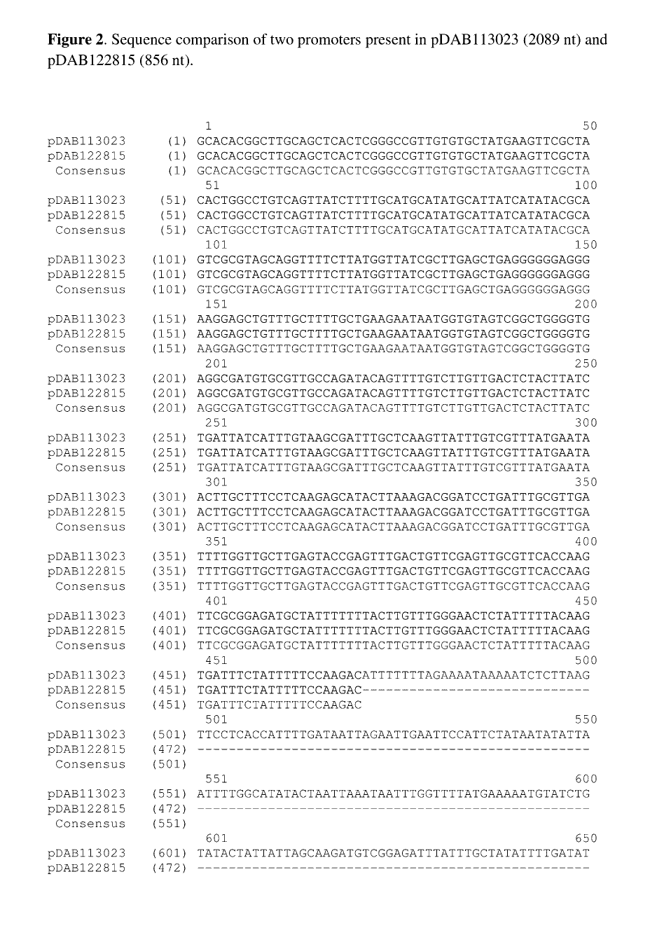

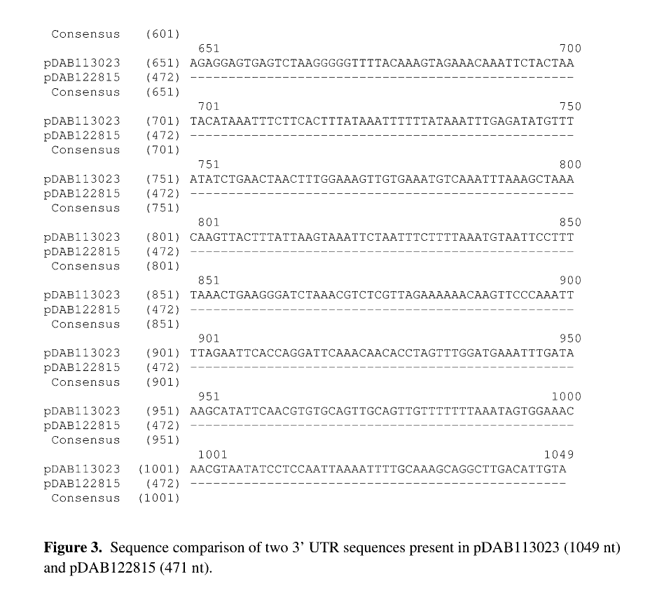

Truncated sequences with repeats removed for the promoter (SEQ ID NO:2) and 3′ UTR (SEQ ID NO:4) from GRMZM2G015295 (SEQ ID NO:5) were used to generate vector pDAB122815 using the same protocol as previously described. Sequence comparisons of the truncated and full length promoters and 3′ UTRs are shown in FIGS. 2 and 3, respectively.

A negative control construct, pDAB101556, was assembled containing an YFP reporter gene instead of the cry34Ab1 gene (FIG. 5) driven by the Zea mays ubiquitin-1 (ZmUbil) promoter v2 and Zea mays Per5 (ZmPer5) 3′ UTR v2 and the same aad-1 expression cassette as present in pDAB113023.

### Example 3: Maize Transformation

### Agrobacterium Culture Initiation

The binary expression vectors were transformed into Agrobacterium tumefaciens strain DAt13192 (RecA deficient ternary strain) (Int'l. Pat. Pub. No. WO2012016222). Bacterial colonies were selected and binary plasmid DNA was isolated and confirmed via restriction enzyme digestion.

Agrobacterium cultures were streaked from glycerol stocks onto AB minimal medium (Gelvin, S., 2006, Agrobacterium Virulence Gene Induction, in Wang, K., ed., Agrobacterium Protocols Second Edition Vol. 1, Humana Press, p. 79; made without sucrose and with 5 g/L glucose and 15 g/L Bacto™ Agar) and incubated at 20° C. in the dark for 3 days. Agrobacterium cultures were then streaked onto a plate of YEP medium (Gelvin, S., 2006, Agrobacterium Virulence Gene Induction, in Wang, K., ed., Agrobacterium Protocols Second Edition Vol. 1, Humana Press, p. 79) and incubated at 20° C. in the dark for 1 day. On the day of an experiment, a mixture of Inoculation medium (2.2 g/L MS salts, 68.4 g/L sucrose, 36 g/L glucose, 115 mg/L L-proline, 2 mg/L glycine, 100 mg/L myo-Inositol, 0.05 mg/L nicotinic acid, 0.5 mg/L pyridoxine HCl, 0.5 mg/L thiamine HCl) and acetosyringone was prepared in a volume appropriate to the size of the experiment. A 1 M stock solution of acetosyringone in 100% dimethyl sulfoxide was added to the Inoculation medium to make a final acetosyringone concentration of 200 μM.

For each construct, 1-2 loops of Agrobacterium from the YEP plate were suspended in 15 ml of the inoculation medium/acetosyringone mixture inside a sterile, disposable, 50 ml centrifuge tube and the optical density of the solution at 600 nm (O.D.₆₀₀) was measured in a spectrophotometer. The suspension was then diluted down to 0.25-0.35 O.D.₆₀₀ using additional Inoculation medium/acetosyringone mixture. The tube of Agrobacterium suspension was then placed horizontally on a platform shaker set at about 75 rpm at room temperature for between 1 and 4 hours before use.

Transformation of Agrobacterium tumefaciens

The experimental constructs were transformed into maize via Agrobacterium-mediated transformation of immature embryos isolated from the inbred line Zea mays c.v. B104. The method used was similar to those published by Ishida et al. (1996) Nature Biotechnol 14:745-750 and Frame et al. (2006) Plant Cell Rep 25: 1024-1034, but with several modifications and improvements as described in Miller (2013) WO 2013090734 A1 to make the method amenable to high-throughput transformation in an industrial setting. An example of a method used to produce a number of transgenic events in maize is given in U.S. Pat. App. Pub. No. US 2013/0157369 A1, beginning with the embryo infection and co-cultivation steps.

### Example 4: To Plant Gene Expression Screening

### Transgene Presence and Copy Number Estimation

Maize plants sampled at the V2-3 leaf stage were screened for transgene presence and their copy number using cry34Ab1 and aad-1 quantitative PCR assays. Total DNA was extracted from 4 leaf punches, using a MagAttract® DNA extraction kit (Qiagen, Valencia, Calif.) as per manufacturer's instructions.

To detect the genes of interest, gene-specific DNA fragments were amplified with TaqMan® primer and probe sets containing a FAM-labeled fluorescent probe for the cry34Ab1 gene and a HEX-labeled fluorescent probe for the endogenous Invertase reference gene control. Primers and probes were used for the cry34Ab1, Invertase, and aad-1 endogenous reference gene amplifications are shown in Table 1.

TABLE 1
Primer and probes sequences for transgene presence and copy number
estimation

| Gene | Oligo Name | Description | SEQ ID NO: |
|---|---|---|---|
| CryAb1 v2 | TQ.8v6.1.F | forward primer | 6 |
| | TQ.8v6.1.R | reverse primer | 7 |
| | TQ.8v6.1.MGB.P | probe | 8 |
| Invertase | InvertaseF | forward primer | 9 |
| | InvertaseR | reverse primer | 10 |
| | InvertaseProbe | probe | 11 |
| AAD1 | AAD1 Forward | forward primer | 12 |
| | AAD1 Reverse | reverse primer | 13 |
| | AAD1 Probe | probe | 14 |

The PCR reactions were carried out in a final volume of 10 μl, containing 5 μl of Roche LightCycler® 480 Probes Master Mix (Roche Applied Sciences, Indianapolis, Ind.); 0.4 μl each of TQ.8v6.1.F, TQ.8v6.1.R, InvertaseF, and InvertaseR primers from 10 μM stocks to a final concentration of 400 nM; 0.4 μl each of TQ.8v6.1.MGB.P Probe and Invertase Probe from 5 μM stocks to a final concentration of 200 nM, 0.1 μl of 10% polyvinylpyrrolidone (PVP) to final concentration of 0.1%; 2 μl of 10 ng/μl genomic DNA and 0.5 μl water. The DNA was amplified in a Roche LightCycler® 480 System under the following conditions: 1 cycle of 95° C. for 10 min; 40 cycles of the following 3-steps: 95° C. for 10 seconds; 58° C. for 35 seconds and 72° C. for 1 second, and a final cycle of 4° C. for 10 seconds. Cry34Ab1 copy number was determined by comparison of Target (gene of interest)/Reference (Invertase gene) values for unknown samples (output by the LightCycler® 480) to Target/Reference values of cry34Ab1v2 copy number controls. The detection of the aad-1 gene was carried out as described above for the cry34Ab1 gene using the invertase endogenous reference gene. aad-1 primer and probe sequences are listed in Table 1.

### To Plant Screening for Gene Expression:

The T₀ plants containing the gene of interest were sampled at V4-5 for Cry34Ab1 and AAD-1 leaf ELISA assays. Four leaf punches were sampled. One ⅛″ stainless steel bead (Hoover Precision Products, Cumming, Ga.) was added to each 1.2 ml tube and 300 μl of extraction buffer (1×PBST) supplemented with 0.5 ml of 500 mM EDTA and 77 mg of DTT powder. The samples were processed in a Genogrinder (SPEX SamplePrep, Metuchen, N.J.) at 1500 rpm for 4 minutes. The samples were centrifuged at 4000 rpm for 2 minutes, and then 300 μl of extraction buffer was added. The samples were processed in Genogrinder for 2 minutes at 1500 rpm and centrifuged at 4000 rpm for 7 minutes. The supernatant was collected and ELISA was performed at different dilutions along with protein standards. The Cry34Ab1 (Agdia, Inc., Elkhart, Ind.) and AAD-1 (Acadia BioSciences Inc., Davis, Calif.) ELISA assays were performed as per the manufacturer's instructions and the ELISA results were expressed either as ng/ml or as parts per million (or ng protein per mg of total plant protein).

Another set of plants were sampled at V4-5 for the entire root mass. The samples were instantly frozen, lyophilized for a week and then ground. The ELISA assays were performed as previously described for the leaf samples. Total root protein estimations were carried out with the Bradford detection method as per the manufacturer's instructions (Pierce, Rockford, Ill.). Root ELISA results were expressed as parts per million or ng protein per mg of total plant protein.

The ELISA results showed root-preferred Cry34Ab1 protein expression in To transgenic events of the construct, pDAB113023 (Tables 2 and 3). No Cry34Ab1 leaf expression was observed in the empty vector, pDAB101556, transgenic events lacking the cry34Ab1 gene. All constructs expressed the aad-1 gene as expected in both roots and leaves.

TABLE 2
T₀ ELISA results showing Cry34Ab1 and AAD-1 transgene expression
in V4-V6 maize leaves of the pDAB113023 construct and the
pDAB101556 control construct.

| | | cry34 | | AAD1 | |
|---|---|---|---|---|---|
| Construct | # of | mean | | mean | |
| name | Events | (ng/ml) | cry34 std | (ng/ml) | AAD1 std |
| pDAB101556 | 3 | 1 | 2 | 247 | 237 |
| pDAB113023 | 10 | 17 | 10 | 345 | 91 |

TABLE 3
ELISA assay resμlts showing Cry34Ab1 and AAD-1 transgene
expression in V4-6 maize roots of the pDAB113023 construct
and the pDAB101556 control construct.

| | # of | Cry34 | | Avg AAD1 | |
|---|---|---|---|---|---|
| Construct | Events | Root | | Root | STD |
| Name | analyzed | (ppm) | STD Root | (ppm) | Root |
| pDAB101556 | 6 | −12 | 8 | 573 | 432 |
| pDAB113023 | 10 | 389 | 324 | 258 | 225 |

### Example 5: T₁ Plant Screening for Transgene Detection and Gene Expression

To plants were reciprocally crossed to Zea mays c.v. B104 non-transgenic transformation lines to obtain T₁ seed. Three to five transgenic lines or events of each of the test regulatory element constructs were advanced for T₁ protein expression studies. Accordingly, 20-30 T₁ seed of each of the events were sown; seedlings were sprayed with Assurell® at the V2-V3 stage of development to kill non-transgenic segregants. All surviving plants were sampled for transgene copy number assays, which were performed as previously described.

For transgene gene expression analysis, plants were sampled at multiple stages of growth and development as follows: leaf (V4, V12 and R3); root (V4 and R3); pollen and silk (all at R1); and husk and kernel (all at R3). All tissues were sampled and then placed in tubes embedded in dry ice; which were then transferred to −80° C. freezers. Frozen tissues for root, kernel, silk samples were lyophilized prior to protein extraction for ELISA. The leaf and husk samples were directly analyzed.

Protein extraction for leaf ELISA was performed as previously described for To samples. Protein extraction for various tissue type ELISA was performed by grinding the lyophilized tissue in a paint shaker for 30 seconds in the presence of eight 0.25 inch ceramic beads (MP Biomedicals, Solon, Ohio). Protein was extracted in 2 ml polypropylene tubes containing enough garnet powder to cover the curved bottom portion of the tubes. The coarsely ground tissue was transferred to the 2 ml tubes to fill up to 0.5 ml mark. One 0.25″ ceramic ball was added to each tube and 0.6 ml of the extraction buffer (200 μl of protease inhibitor cocktail [Research Products International, Solon, Ohio], 200 μl of 500 mM EDTA, 15.5 mg DTT powder and PBST to 20 ml). All tubes were kept on ice for 10 minutes and then processed for 45 seconds in the Genogrinder. Next, 40 μl of 10% Tween 20 and 300 μl extraction buffer was added, followed by grinding for 45 seconds. The tubes were centrifuged at 13,000 rpm for 7-14 minutes. The supernatant was transferred to a new tube to collect the extract, which was diluted the as previously described for ELISA assays.

pDAB113023 T₁ plants exhibited high expression of the Cry34Ab1 protein in roots (Table 4). Leaf expression of pDAB113023 remained lower (Table 4). The AAD-1 expression was in the expected ranges.

No Cry34Ab1 leaf expression was observed in the negative control vector, pDAB101556, which lacked the cry34Ab1 gene. Lower expression was observed in different stages of leaf development, husk, kernel and pollen. All constructs exhibited AAD-1 expression in all tissue types.

TABLE 4
Cry34Ab1 reporter gene expression in different tissue types of
pDAB113023 transgenic events controlled by full-length versions of the promoter (SEQ ID
NO: 1) and corresponding native 3′ UTR (SEQ ID NO: 3). pDAB101556 is a negative control
construct containing a yfp reporter gene cassette instead of that of cry34Ab1. The aad-1
selectable marker cassette was identical in both pDAB113023 and pDAB101556 constructs.

| | | | | Mean | | Mean | |
|---|---|---|---|---|---|---|---|
| | | # Events | # plants | Cry34Ab1 | Cry34Ab1 | AAD-1 | AAD-1 |
| Construct | Tissue | analyzed | analyzed | (ppm) | STD | (ppm) | STD |
| pDAB101556 | Leaf (V4) | 1 | 14 | 1 | 0 | 773 | 133 |
| pDAB113023 | Leaf (V4) | 3 | 45 | 188 | 20 | 824 | 175 |
| pDAB101556 | Leaf (V12) | 1 | 3 | 1 | 0 | 1381 | 166 |
| pDAB113023 | Leaf (V12) | 3 | 18 | 220 | 43 | 1488 | 550 |
| pDAB101556 | Leaf (R3) | 1 | 3 | 1 | 0 | 6042 | 1728 |
| pDAB113023 | Leaf (R3) | 3 | 18 | 310 | 100 | 5628 | 1499 |
| pDAB101556 | Root (V4) | 1 | 3 | 1 | 0 | 3307 | 640 |
| pDAB113023 | Root (V4) | 2 | 9 | 1641 | 227 | 2817 | 746 |
| pDAB113023 | Pollen | 2 | 7 | 407 | 80 | 5063 | 1746 |
| pDAB113023 | Husk | 2 | 8 | 915 | 206 | 5837 | 1032 |
| pDAB113023 | Kernel | 1 | 1 | 852 | 0 | 5223 | 0 |

The repeat-deleted versions of the promoter and 3′ UTR present in construct pDAB122815 exhibited root and other tissue type expression in similar range as compared to the full-length versions present in construct pDAB113023 (Table 5).

TABLE 5
Cry34Ab1 reporter gene expression in different tissue types of
pDAB122815 transgenic events controlled by repeat-deleted versions of the promoter
(SEQ ID NO: 2) and corresponding 3′ UTR (SEQ ID NO: 4). pDAB101556 is a
negative control construct containing a yfp reporter gene cassette instead of that of
cry34Ab1. The aad-1 selectable marker cassette was identical in both pDAB122815
and pDAB101556 constructs.

| | | | | Cry34Ab1 | | AAD-1 | |
|---|---|---|---|---|---|---|---|
| Construct | | Total | Total | Mean | Cry34Ab1 | Mean | AAD-1 |
| Name | Tissue | Events | Plants | (ng/mg) | STD | (ng/mg) | STD |
| pDAB101556 | Leaf V4 | 1 | 9 | 1 | 4 | 79 | 71 |
| pDAB122815 | Leaf V4 | 5 | 62 | 135 | 29 | 232 | 156 |
| pDAB101556 | Leaf V12 | 1 | 5 | 2 | 3 | 909 | 140 |
| pDAB122815 | Leaf V12 | 5 | 21 | 93 | 26 | 920 | 171 |
| pDAB101556 | Leaf R3 | 1 | 5 | 4 | 6 | 969 | 322 |
| pDAB122815 | Leaf R3 | 5 | 22 | 73 | 12 | 1035 | 205 |
| pDAB122815 | Roots V4 | 3 | 9 | 1356 | 267 | 4163 | 710 |
| pDAB122815 | Roots R3 | 5 | 15 | 879 | 268 | 3017 | 1615 |

What is claimed is:

1. A gene expression cassette comprising a promoter operably linked to a transgene, wherein the promoter comprises a polynucleotide that hybridizes under stringent conditions to a polynucleotide probe comprising a sequence identity of at least 90% to a complement of SEQ ID NO:1.

2. The gene expression cassette of claim 1, wherein the polynucleotide has at least 90% sequence identity to SEQ ID NO:1.

3. The gene expression cassette of claim 1, wherein the polynucleotide has at least 90% sequence identity to SEQ ID NO:2.

4. The gene expression cassette of claim 1, wherein the operably linked transgene encodes a polypeptide or a non-coding RNA.

5. The gene expression cassette of claim 1, wherein the transgene is selected from the group consisting of insecticidal resistance transgene, herbicide tolerance transgene, nitrogen use efficiency transgene, water use efficiency transgene, nutritional quality transgene, DNA binding protein transgene, and selectable marker transgene.

6. The gene expression cassette of claim 1 further comprising a 3′-untranslated region.

7. The gene expression cassette of claim 6, wherein the 3′-untranslated region has at least 90% sequence identity to SEQ ID NO:3 or SEQ ID NO:4.

8. A recombinant vector comprising the gene expression cassette of claim 1.

9. The recombinant vector of claim 8, wherein the vector is selected from the group consisting of a plasmid, a cosmid, a bacterial artificial chromosome, a virus, and a bacteriophage.

10. A transgenic cell comprising the gene expression cassette of claim 1.

11. The transgenic cell of claim 10, wherein the transgenic cell is a transgenic plant cell and wherein the transgenic cell is a transiently transformed transgenic cell.

12. A transgenic plant comprising the transgenic plant cell of claim 11.

13. The transgenic plant of claim 12, wherein the transgenic plant is a monocotyledonous plant or dicotyledonous plant.

14. The transgenic plant of claim 13, wherein the monocotyledonous plant is selected from the group consisting of a maize plant, a rice plant, and a wheat plant.

15. A transgenic seed from the transgenic plant of claim 12.

16. The gene expression cassette of claim 1, wherein the promoter is a tissue-preferred promoter.

17. The gene expression cassette of claim 1, wherein the tissue-preferred promoter is a root tissue-preferred promoter.

18. The gene expression cassette of claim 1, wherein the promoter comprises a polynucleotide sequence of nucleotides 1-2,089 of SEQ ID NO:1.

19. A transgenic cell comprising a synthetic polynucleotide that hybridizes under stringent conditions to a polynucleotide probe comprising a sequence identity of at least 90% to a complement of SEQ ID NO:1.

20. The transgenic cell of claim 19, wherein the synthetic polynucleotide has at least 90% sequence identity to SEQ ID NO:1.

21. The transgenic cell of claim 19, wherein the synthetic polynucleotide has at least 90% sequence identity to SEQ ID NO:2.

22. The transgenic cell of claim 19, wherein the transgenic cell is a transgenic plant cell.

23. The transgenic cell of claim 22, wherein the transgenic plant cell is produced by a plant transformation method.

24. The transgenic cell of claim 23, wherein the plant transformation method is selected from the group consisting of an Agrobacterium-mediated transformation method, a biolistics transformation method, a silicon carbide transformation method, a protoplast transformation method, and a liposome transformation method.

25. A transgenic plant comprising the transgenic plant cell of claim 22.

26. The transgenic plant of claim 25, wherein the transgenic plant is a monocotyledonous plant or dicotyledonous plant.

27. The transgenic plant of claim 26, wherein the monocotyledonous plant is selected from the group consisting of a maize plant, a rice plant, and a wheat plant.

28. A transgenic seed from the transgenic plant of claim 25.

29. The transgenic cell of claim 19, wherein the promoter is a tissue-preferred promoter.

30. The transgenic cell of claim 19, wherein the tissue-preferred promoter is a root tissue-preferred promoter.


SEQUENCE LISTING (14 sequences)

SEQ ID NO: 1 - DNA, 2089 bp, Zea mays
tcaggtatgagtgctacttttcaatgcatcaagaagttcttttgttggaggggtatgaagacagaattggagttgtttgttaggcagtgtggaatatgccggaaagctaagattgagaggcattatcctgctgggctgcttcagccactacctgttccagcaggggcatgggaggacatttctatggatttcattgaaaaactgccccaagttagaaggttttcacacaattattatggtggtggacaaatttcaaagtatacccacttctttccacttaagcatcctttcacaactcaaggtgtggcacaagtgatacttgataatgtggttaagcttcatgggctccctaagtcaattgtgtgtgatagagataagatcttcacaagtaacttctagaatcatttgttcaagttgttggagatgaagttggctcttagcaccgtctaccatcctcaaacatatggtcaaagtgaagagtgaatcaaagtctagagatgtatcttagatgtgctgcttctgaaaatccacagaaatggaaaacttggttggctttggcagagttttggtataacacatcatatcatgtgtctttgagatgttctcctttcaaggctttgtatggatatgatgcttctatgatctggcacgatctatgaagggtgatgaagatgtggtttctagagatgtggtgcaacagatagcagtgcatagttctatgctcaaggaacatttgacgaaagtccaacaaaggtttaaacattatgctgatatgagaaggttgcctagggatttcaaattgaagaagaggtgttgctcaagttatagcctacacacaacagtctgtggttagcaaacaatgtccaaagttttcttttcgatattttggaccatacaaggtgttggagaaaattgggaagtggcttacaagttggatctaccagcagactctcaaatacacctagtgtttcatgtgtcacagctaaagccctttgtgcctcgttatacaccggtgttcaagaaccttcctgctatggtggatctagagaatgttgagccaggtaaagtgctagaccgaagattggttaagaaggggaatgcaaccattactcaggttctggttaggtggaccagtccatctccagatcttgcaacatgggaagattaccatgtcttgaaggctcgttttctagctgttcttgcttgaggagaagcaagatttgaagggggatgtaaggcctggtgatattgctaccttttggtgttagggtttgggtagtattttgataatattttgtttgtaatccattagtggaataatgaccggtttctagaagtagaaaggggtattacactatcgtggtagtaggtgagttcatccgtccaatctgtgccactggaagagaggacagggaggagtggaggtgcgacgaccaatggtggtggcggcgacggctggaagaggagagcaaggagaatgagagaggatgaagcatgaatcgggctgtgtgacgggagagcaggtatttttaattggaccactgtttattttgagaaatcggagggtgtttttgcgaaccaagtctcgagatgtgtcagtatatgtataacgtgtttggtttgaggaataagttagtctattatcttctcactcctcactttcttgtttggtttgtaaaatataatggttttatccgtcactatttcctttatttggtaataattagtgataattaaaaaaataagttcatttcactaaatttataaaataaactcataatgcacgaacttatataaaatagattgattccatcattggtcaccaatgtctcagatccgcgcatctcacctgacgtcttcacccaccaacggcactccggtatggcgactcaccaaccccccaccaatcccccgcccagatccgacggccgctaatcgccgcgtccgcaaaccaacggtctggcgccgtcgtctcttcccataaaaccctccccacccctgcctcccaaccgcgccgtcttctccctcactcccgttccatttccccatctcccagatccaattcgcgagttctccctcctctgccgcc

SEQ ID NO: 2 - DNA, 857 bp, Zea mays
gtaaggcctggtgatattgctaccttttggtgttagggtttgggtagtattttgataatattttgtttgtaatccattagtggaataatgaccggtttctagaagtagaaaggggtattacactatcgtggtagtaggtgagttcatccgtccaatctgtgccactggaagagaggacagggaggagtggaggtgcgacgaccaatggtggtggcggcgacggctggaagaggagagcaaggagaatgagagaggatgaagcatgaatcgggctgtgtgacgggagagcaggtatttttaattggaccactgtttattttgagaaatcggagggtgtttttgcgaaccaagtctcgagatgtgtcagtatatgtataacgtgtttggtttgaggaataagttagtctattatcttctcactcctcactttcttgtttggtttgtaaaatataatggttttatccgtcactatttcctttatttggtaataattagtgataattaaaaaaataagttcatttcactaaatttataaaataaactcataatgcacgaacttatataaaatagattgattccatcattggtcaccaatgtctcagatccgcgcatctcacctgacgtcttcacccaccaacggcactccggtatggcgactcaccaaccccccaccaatcccccgcccagatccgacggccgctaatcgccgcgtccgcaaaccaacggtctggcgccgtcgtctcttcccataaaaccctccccacccctgcctcccaaccgcgccgtcttctccctcactcccgttccatttccccatctcccagatccaattcgcgagttctccctcctctgccgcc

SEQ ID NO: 3 - DNA, 1049 bp, Zea mays
gcacacggcttgcagctcactcgggccgttgtgtgctatgaagttcgctacactggcctgtcagttatcttttgcatgcatatgcattatcatatacgcagtcgcgtagcaggttttcttatggttatcgcttgagctgaggggggagggaaggagctgtttgcttttgctgaagaataatggtgtagtcggctggggtgaggcgatgtgcgttgccagatacagttttgtcttgttgactctacttatctgattatcatttgtaagcgatttgctcaagttatttgtcgtttatgaataacttgctttcctcaagagcatacttaaagacggatcctgatttgcgttgattttggttgcttgagtaccgagtttgactgttcgagttgcgttcaccaagttcgcggagatgctatttttttacttgtttgggaactctatttttacaagtgatttctatttttccaagacatttttttagaaaataaaaatctcttaagttcctcaccattttgataattagaattgaattccattctataatatattaattttggcatatactaattaaataatttggttttatgaaaaatgtatctgtatactattattagcaagatgtcggagatttatttgctatattttgatatagaggagtgagtctaagggggttttacaaagtagaaacaaattctactaatacataaatttcttcactttataaattttttataaatttgagatatgtttatatctgaactaactttggaaagttgtgaaatgtcaaatttaaagctaaacaagttactttattaagtaaattctaatttcttttaaatgtaattccttttaaactgaagggatctaaacgtctcgttagaaaaaacaagttcccaaattttagaattcaccaggattcaaacaacacctagtttggatgaaatttgataaagcatattcaacgtgtgcagttgcagttgtttttttaaatagtggaaacaacgtaatatcctccaattaaaattttgcaaagcaggcttgacattgta

SEQ ID NO: 4 - DNA, 471 bp, Zea mays
gcacacggcttgcagctcactcgggccgttgtgtgctatgaagttcgctacactggcctgtcagttatcttttgcatgcatatgcattatcatatacgcagtcgcgtagcaggttttcttatggttatcgcttgagctgaggggggagggaaggagctgtttgcttttgctgaagaataatggtgtagtcggctggggtgaggcgatgtgcgttgccagatacagttttgtcttgttgactctacttatctgattatcatttgtaagcgatttgctcaagttatttgtcgtttatgaataacttgctttcctcaagagcatacttaaagacggatcctgatttgcgttgattttggttgcttgagtaccgagtttgactgttcgagttgcgttcaccaagttcgcggagatgctatttttttacttgtttgggaactctatttttacaagtgatttctatttttccaagac

SEQ ID NO: 5 - DNA, 5527 bp, Zea mays
tcaggtatgagtgctacttttcaatgcatcaagaagttcttttgttggaggggtatgaagacagaattggagttgtttgttaggcagtgtggaatatgccggaaagctaagattgagaggcattatcctgctgggctgcttcagccactacctgttccagcaggggcatgggaggacatttctatggatttcattgaaaaactgccccaagttagaaggttttcacacaattattatggtggtggacaaatttcaaagtatacccacttctttccacttaagcatcctttcacaactcaaggtgtggcacaagtgatacttgataatgtggttaagcttcatgggctccctaagtcaattgtgtgtgatagagataagatcttcacaagtaacttctagaatcatttgttcaagttgttggagatgaagttggctcttagcaccgtctaccatcctcaaacatatggtcaaagtgaagagtgaatcaaagtctagagatgtatcttagatgtgctgcttctgaaaatccacagaaatggaaaacttggttggctttggcagagttttggtataacacatcatatcatgtgtctttgagatgttctcctttcaaggctttgtatggatatgatgcttctatgatctggcacgatctatgaagggtgatgaagatgtggtttctagagatgtggtgcaacagatagcagtgcatagttctatgctcaaggaacatttgacgaaagtccaacaaaggtttaaacattatgctgatatgagaaggttgcctagggatttcaaattgaagaagaggtgttgctcaagttatagcctacacacaacagtctgtggttagcaaacaatgtccaaagttttcttttcgatattttggaccatacaaggtgttggagaaaattgggaagtggcttacaagttggatctaccagcagactctcaaatacacctagtgtttcatgtgtcacagctaaagccctttgtgcctcgttatacaccggtgttcaagaaccttcctgctatggtggatctagagaatgttgagccaggtaaagtgctagaccgaagattggttaagaaggggaatgcaaccattactcaggttctggttaggtggaccagtccatctccagatcttgcaacatgggaagattaccatgtcttgaaggctcgttttctagctgttcttgcttgaggagaagcaagatttgaagggggatgtaaggcctggtgatattgctaccttttggtgttagggtttgggtagtattttgataatattttgtttgtaatccattagtggaataatgaccggtttctagaagtagaaaggggtattacactatcgtggtagtaggtgagttcatccgtccaatctgtgccactggaagagaggacagggaggagtggaggtgcgacgaccaatggtggtggcggcgacggctggaagaggagagcaaggagaatgagagaggatgaagcatgaatcgggctgtgtgacgggagagcaggtatttttaattggaccactgtttattttgagaaatcggagggtgtttttgcgaaccaagtctcgagatgtgtcagtatatgtataacgtgtttggtttgaggaataagttagtctattatcttctcactcctcactttcttgtttggtttgtaaaatataatggttttatccgtcactatttcctttatttggtaataattagtgataattaaaaaaataagttcatttcactaaatttataaaataaactcataatgcacgaacttatataaaatagattgattccatcattggtcaccaatgtctcagatccgcgcatctcacctgacgtcttcacccaccaacggcactccggtatggcgactcaccaaccccccaccaatcccccgcccagatccgacggccgctaatcgccgcgtccgcaaaccaacggtctggcgccgtcgtctcttcccataaaaccctccccacccctgcctcccaaccgcgccgtcttctccctcactcccgttccatttccccatctcccagatccaattcgcgagttctccctcctctgccgccatggcgctctctgtggagaagacctcgtctggacgggagtacaaggtcaaggatctctcgcaggcggacttcggccgcctcgagattgagctggccgaggtcgaaatgcccggcctcatggcgtgccgcgccgagttcggcccgtccaagcccttcgccggcgctaggatctcggggtctctccacatgaccatccagaccgccgtcctcatcgagaccctcaccgcgctcggcgccgaggtccgctggtgctcctgcaacatcttctccacgcaggaccacgccgccgccgccatcgcgcgcgactcggccgccgtgttcgcctggaagggggagaccctcgaggagtactggtggtgcaccgagcgctgcctcgactggggcgaggcgggcggccccgacctcatcgtcgacgacggcggcgacgccacgctgctcatccacgagggtgtcaaggccgaggaggattacgagaagaccggcaagatccccgacccggagtccaccgacaacgctgagttcaagatcgtgctcaccatcatccgcgacgggctcaaggctgaccccaagaagtaccgcaagatgaaggagaggcttgtcggcgtctctgaggagaccaccacgggtgtcaagaggctctaccagatgcaggagaccggcgccctcctcttccctgccattaacgtcaacgattccgtcaccaagagcaaggtgattcttgcttgcctttctttggatccatttcctttcagatctgacgttcttgttgtgtggatctagatcctgcatgactagcagtgtgagatttacttgtttattgatgccgctgctttatatctcagtatctcactgctatggttaaactcttgtgtatcttcagattcggctgctagacgttcttgtttattggttgctagatggttagtaatttttacctagtatttggatcttcagatctctgttagacaggacctgatttttgggattgattgtagaaggcgaagcatctcgggatattttaggtcttttttgtgtagccagattcggctgctgttatatgtttggttccagttacttgacctaaatcgcattgttgttgttgtggatttagcgtttctcattgttgttgttttctgttggatctggatcctggtctatggtgttgcttatctatcggtcaaaataaaaattattgttagctacttcaaatattttatacgtgtttggtccatgtgatgtgttttgcatgtttggtggatttgacgacactatgtcatcatagctaataatttcttgcaccctgtttacttagtctgtatgtagatttgacacgtattttcagatcaatacatgtttcggttagtctatgtatttttagtgtcttgctgcacattttctattcttaactacgaatattgaactcttaattacaaatatttttcttgtaaacagtttgacatgttgctagtaccgaaattcaactattaagactaactaaattgattgatatggttctcctctgaacagtttgacaacctgtatggttgccgccactcgctccctgatggtctgatgagggccactgacgttatgatcgccggaaaggttgccgtggtctgcggatacggtgatgtcggcaagggttgtgctgctgccctcaagcaggctggtgcccgtgtcattgtgaccgagatcgaccccatctgtgccctccaggctctgatggagggtcttcaggtccttcccttggaggacgttgtctctgaagctgacatcttcgtgaccaccactggcaacaaggatatcatcatggttgaccacatgaggaagatgaagaacaatgccattgtctgcaacattggccactttgacaatgaaattgatatgctcggccttgagacctaccctggcgtcaagcgcatcaccatcaagccccagactgaccgctgggtgttccccgagaccaacactggcatcattgtccttgctgagggtcgcctgatgaaccttgggtgtgctactggccatcctagctttgtcatgtcctgctcattcactaaccaggtaagcactggaattggtggtattgccatatgtaaactgattggctagcaatgccacattactgagttgtttactcttgacttgatgatgcaggtcattgcccaacttgaactgtggaaggagaagagctctggcaagtatgagaagaaggtgtatgtgctccccaagcaccttgatgagaaggttgctgctctccacttgggcaagcttggtgccaagctgaccaagctcaccaagtctcaggccgactacatcagcgtgccgatcgagggtccctacaagcctgcccactaccggtactaggcacacggcttgcagctcactcgggccgttgtgtgctatgaagttcgctacactggcctgtcagttatcttttgcatgcatatgcattatcatatacgcagtcgcgtagcaggttttcttatggttatcgcttgagctgaggggggagggaaggagctgtttgcttttgctgaagaataatggtgtagtcggctggggtgaggcgatgtgcgttgccagatacagttttgtcttgttgactctacttatctgattatcatttgtaagcgatttgctcaagttatttgtcgtttatgaataacttgctttcctcaagagcatacttaaagacggatcctgatttgcgttgattttggttgcttgagtaccgagtttgactgttcgagttgcgttcaccaagttcgcggagatgctatttttttacttgtttgggaactctatttttacaagtgatttctatttttccaagacatttttttagaaaataaaaatctcttaagttcctcaccattttgataattagaattgaattccattctataatatattaattttggcatatactaattaaataatttggttttatgaaaaatgtatctgtatactattattagcaagatgtcggagatttatttgctatattttgatatagaggagtgagtctaagggggttttacaaagtagaaacaaattctactaatacataaatttcttcactttataaattttttataaatttgagatatgtttatatctgaactaactttggaaagttgtgaaatgtcaaatttaaagctaaacaagttactttattaagtaaattctaatttcttttaaatgtaattccttttaaactgaagggatctaaacgtctcgttagaaaaaacaagttcccaaattttagaattcaccaggattcaaacaacacctagtttggatgaaatttgataaagcatattcaacgtgtgcagttgcagttgtttttttaaatagtggaaacaacgtaatatcctccaattaaaattttgcaaagcaggcttgacattgtattgtagacggaagcctgacatt

SEQ ID NO: 6 - DNA, 17 bp, Artificial Sequence
  Cry34Ab1 v2 forward primer
gccataccctccagttg

SEQ ID NO: 7 - DNA, 23 bp, Artificial Sequence
  Cry34Ab1 v2 reverse primer
gccgttgatggagtagtagatgg

SEQ ID NO: 8 - DNA, 18 bp, Artificial Sequence
  Cry34Ab1 v2 probe
ccgaatccaacggcttca

SEQ ID NO: 9 - DNA, 18 bp, Artificial Sequence
  nvertase forward primer
tggcggacgacgacttgt

SEQ ID NO: 10 - DNA, 19 bp, Artificial Sequence
  nvertase reverse primer
aaagtttggaggctgccgt

SEQ ID NO: 11 - DNA, 21 bp, Artificial Sequence
  InvertaseProbe
cgagcagaccgccgtgtactt

SEQ ID NO: 12 - DNA, 20 bp, Artificial Sequence
  AAD1 Forward Primer
tgttcggttccctctaccaa

SEQ ID NO: 13 - DNA, 22 bp, Artificial Sequence
  AAD1 Reverse Primer
caacatccatcaccttgactga

SEQ ID NO: 14 - DNA, 24 bp, Artificial Sequence
  AAD1 Probe
cacagaaccgtcgcttcagcaaca